(19) United States
(12) Patent Application Publication
(10) Pub. No.: US 2002/0147326 A1
(43) Pub. Date: Oct. 10, 2002

(54) Hexameric fusion proteins and uses therefor

(75) Inventors: Margery Ann Chaikin, Wayne, PA (US); Sally Doreen Patricia Lyn, West Chester, PA (US); Raymond W. Sweet, Bala Cynwyd, PA (US); Alemseged Truneh, West Chester, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 09/845,899

(22) Filed: Apr. 30, 2001

Related U.S. Application Data
(63) Continuation of application No. 09/202,346, filed on Jan. 13, 1999, now abandoned.
Application No. 09/202,346 is a 371 of international application No. PCT/US97/12599, filed on Jun. 13, 1997.
(60) Provisional application No. 60/019,934, filed on Jun. 14, 1996.
(60) Provisional application No. 60/043,948, filed on Feb. 19, 1997.
(60) Provisional application No. 60/038,915, filed on Feb. 21, 1997.

(51) Int. Cl. A61K 39/395; C07H 21/04; C12P 21/04; C07K 16/46; C12N 5/06

(52) U.S. Cl. 536/23.5; 435/326; 435/320.1; 424/178.1; 530/389.1; 435/69.7

(57) ABSTRACT

An hexameric fusion protein containing a dimeric binding protein provided with a tailpiece from an IgA antibody is described. This fusion protein is useful in therapeutics and vaccines, but is particularly well suited for applications for which the binding protein from which it is derived is unsatisfactory because of low binding affinity or for applications where multivalency is desired. These applications include diagnostics, binding assays and screening assays.

### RELATED APPLICATION

This application claims benefit of U.S. Provisional patent applications numbered 60/019,934 filed Jun. 14, 1996, 60/043,948 filed Feb. 19, 1997 and 60/038,915 filed Feb. 21, 1997.

### BACKGROUND OF THE INVENTION

IgM and IgA are the two classes of human antibodies that form homo-oligomeric structures. By far the most extensively studied of these is IgM.

The classical view of IgM structure is as a pentamer in combination with a single copy of a second protein, the J-chain that becomes associated with IgM during its assembly and export. This J-chain can covalently associate with IgM through the formation of a disulfide bond between a cysteine residue in the J chain and a cysteine residue in a short 18 amino acid extension. designated μtp, from the canonical C-terminal constant region of the heavy chain. This cysteine residue appears to be required for the formation of the IgM pentamer in association with the J-chain [A. C. Davis et al., EMBO J., 8(9):2519-2526 (1989)]. However, a hexameric form of IgM, devoid of the J-chain, was described and characterized over two decades ago and has recently been characterized in more detail in terms of its biochemical and potential biological activities [reviewed in Brewer et al., Immunology Today, 15:165-168 (1994)]. The production of oligomeric IgG proteins has been achieved by addition of the 18 amino acid IgM tailpiece segment (utp) to the αtp corresponding C-termini end of the Cγ3 region of the IgG1-4 proteins by DNA recombinant technology [R. I. F. Smith and S. L. Morrison, Biotechnolocy, 12:683-688 (1994); R. I. F. Smith et al., J. Immunol., 154:2226-2236 (1995)].

Human IgA also has an 18 amino acid tailpiece segment (αtp) which bears some sequence homology to utp. In man, there are two α constant region loci which encode distinct sequences, but the tailpiece regions for the α1 and α2 regions are quite similar, or in some cases reported to be identical [Sequences of Proteins of Immunological Interest, fifth edition, EA Kabat et al., Vol. 1, U.S. Department of Health and Human Services, NIH publication no.91-3242, (1991)]. However, unlike IgM, IgA occurs most frequently as a monomer antibody, similar to the IgG subclasses, or as a dimer antibody plus one molecule of J-chain [Mestecky and Kilian, Methods in Enzymology, 116:37-75 (1985); T. B. Tomasi, Immun. Today, 13:416-418 (1992)]. Higher oligomers/aggregates of IgA are reported [Mestecky and Kilian, cited above], but these are poorly characterized components in complex mixtures containing other proteins interactive with IgA. Recombinant IgA has been expressed in the presence and absence of theJ chain (Bruggemann et al., J. Exp. Med., 166:1351-1361 (1987); Morton et al., J. Immunol., 151:4743-4752 (1993); Carayannopoulos et al., Proc. Natl Acad Sci, USA, 91:8348-8352 (1994); Terskikh et al., Mol. Immunol., 31:1313-1319 (1994)]. The IgA proteins produced in the absence of the J chain were monomeric or dimeric forms by nonreducing SDS/PAGE and appeared as dimers in solution. In one study (Carayannopoulos et al., above), the co-expression of the J-chain led to formation of disulfide linked IgA dimers together with J chain.

The CD28 receptor, a member of the immunoglobulin superfamily of molecules (IgSF) [A. F. Williams and A. N. Barclay, Annu. Rev. Immunol., 6:381-405 (1988)], is a 44 kDa homodimer glycoprotein expressed on the surface of T-lineage cells including thymocytes and peripheral T cells in the spleen, lymph node and peripheral blood. CD28 interacts with two different counter-receptors CD80 (also known as B7 and B7.1) [P. S. Linsley et al., Proc. Natl. Acad. Sci. USA, 87(13):5031-5035 (1990); G. J. Freeman et al., J. Exp. Med., 174(3):625-631 (1991)] and CD86 (also called B7.2 and B70) [M. Azuma et al., Nature, 366(6450):76-79 (1993); G. J. Freeman et al., J. Exp. Med., 178(6):2185-2192 (1993); G. J. Freeman et al., Science, 262(5135):909-911 (1993)], expressed on antigen presenting cells (APCs), to deliver crucial co-stimulatory signals for sustained activation of T cells, through its association via the cytoplasmic domain with PI3-kinase [F. Pages et al., Nature, 369(6478):327-329 (1994); P. H. Stein et al., Molecul. & Cell. Biol., 14(5):3392-3402 (1994)] and other signalling pathways [K. E. Truitt et al., J. Immunol., 155:4702-4710 (1995); J. A. Nunes et al., J. Biol. Chem., 271(3): 1591-1598 (1996); H. Schweider et al., Eur. J. Immunol., 25:1044-1050 (1995)]. Both CD80 [P. S. Linsley et al., J. Exp. Med., 174(3):561-569 (1991)] and CD86 [Azuma et al., cited above; Freeman et al., 1993, cited above; Freeman et al., 1993, cited above] also recognize CTLA-4 [J. F. Brunet et al., Nature, 328(6127):267-270 (1987)], a homolog of CD28, expressed transiently and at low receptor density on activated CD8⁺ and CD4⁺ T cells.

Antagonism of CD28 interactions with the CD80 or CD86 counter-receptors using CTLA4-Ig fusion proteins or antibodies directed against CD80 and CD86 inhibits T cell activation in vitro, suppresses humoral and cellular immune responses in vivo, inhibits graft rejection and the progression of autoimmune diseases in vivo [reviewed in J. A. Bluestone, Immunity, 2:555-559 (1995); Harlan et al., Clin. Immunol. and Immunopath., 75(2):99-111 (1995)]. Thus, CD28 is a target for development of immunosuppressive agents. To identify small molecule antagonists, a rapid and reproducible assay is desirable for the screening of synthetic compounds, natural products, and peptides. Particularly desirable is a protein based assay which would isolate the receptor and its counter-receptor from interference by other components of cell-based assays, and which is additionally adaptable to automation. The affinity of the interaction of CD28 with both counter receptors is quite low [P. S. Linsley et al., Immunity, 1:793-801 (1994)], with an approximate Kd of 200 nM for the binding of a soluble CD80-Ig fusion protein to an immobilized CD28-Ig fusion protein [P. S. Linsley et al., J. Exp. Med., 173(3):721-730 (1991)]. This low affinity hampers development of a sensitive protein binding assays amenable to screening many compounds.

What is needed is a method for increasing the avidity of binding proteins, particularly those with low affinity, for use in screening and diagnostic assays, therapeutics, and vaccines.

### SUMMARY OF THE INVENTION

In one aspect, the present invention provides a hexameric fusion protein which provides increased binding activity as compared to the protein from which it is derived and methods of making same. This fusion protein is particularly useful in binding assays and may be readily purified.

The hexameric fusion protein of the invention contains a dimeric binding protein and a tailpiece (αtp) characterized by the activity of the tailpiece from the C-terminus of the heavy chain of an IgA antibody. In one embodiment, the binding protein is a natively dimeric binding protein or a functional fragment thereof. In another embodiment, the binding protein is recombinantly engineered to have a dimeric form. This is preferably achieved by fusion of a protein fragment which contains the extracellular domain of a selected binding protein to an Fc fragment. These binding proteins, when provided with the αtp, assemble into homo- or hetero-hexamers.

In yet another aspect, the present invention provides a polynucleotide sequence encoding a stable hexameric fusion protein of the invention.

In a further aspect, the present invention provides a vector comprising the above-described polynucleotide sequence and a sequence controlling expression of the fusion protein in a selected host cell.

In still another aspect, the present invention provides a recombinant host cell containing the above-described vector.

In a further aspect, the present invention provides methods of producing and purifying a stable hexameric fusion protein by providing a host cell containing the stable hexameric fusion protein of the invention, recovering the stable hexameric fusion protein, and purifying the recovered protein. The strands of the fusion protein are preferably co-produced and assembled in the host cell.

In still a further aspect, the present invention provides a pharmaceutical composition containing a stable hexameric fusion protein or a DNA sequence encoding the stable hexameric fusion protein of the invention and a pharmaceutically acceptable carrier.

In yet another aspect, the present invention provides for screening for ligands to a hexameric fusion protein of the invention. Also provided are assays for inhibitors of hexameric binding protein/ligand interaction.

Other aspects and advantages of the present invention are described further in the following detailed description of the preferred embodiments thereof.

### BRIEF DESCRIPTION OF THE DRAWINGS

FIG. 1 is a schematic representation of the hexameric CD80-Igαtp protein of the invention. The regions of the molecule corresponding to the CD80 extracellular domain, the IgG1 hinge, CH2, and CH3 domains, and the αtp segment are indicated. The letter “S” in the diagram indicates the positions of predicted disulfide bonds between cysteine residues.

FIG. 2 is a plasmid map illustrating the expression construct for the CD80-Igαtp protein of the invention. The plasmid is 7,167 base pairs in size. Beginning at residue 1 in a clockwise manner: “cmv pro” is the major late CMV promoter for transcription of the downstream CD80-Igαtp coding sequence; “CD80” encodes the signal peptide and extracellular domain of human CD80; “Fc” encodes the hinge, CH2, and CH3 regions of human IgG1. “αtp” encodes the human αtp segment; “BGH” is the polyadenylation signal region from the bovine growth hormone gene; “betaglobin” is the mouse major b-globin promoter; “dhfr” encodes the mouse dhfr (dihydrofolate reductase) protein; “SV40” is the SV40 early polyadenylation region; and “ori” and “amp” are the bacterial origin of replication and beta lactamase gene, respectively, from the common cloning plasmid pBR322. The corresponding plasmids CD86Fcαtplink and CTLA4Fcαtplink were constructed for the expression of the CD86-Igαtp and CTLA4-Igαtp proteins (see FIGS. 5 and 6).

FIGS. 3A-3H is the complete DNA sequence of the CD80Fcαtplink plasmid [SEQ ID NO:1] shown in FIG. 2.

FIGS. 4A-4D is the DNA and encoded protein [SEQ ID NOS: 2 and 3] sequences for the CD80-Igαtp region in the vector CD80-Fcαtplink. Bolded regions show restriction sites for reference to FIG. 2 and the initiation codon, mature processing site, hinge region, and C-terminal αtp segment.

FIGS. 5A-5B is the DNA and encoded protein sequences [SEQ ID NOS. 4 and 5] for the extracellular domain of CD86 in the vector CD86Fcαtplink. The sequence outside of the Kpn I and Eag I sites is the same as for CD80Fcαtplink (see FIGS. 3A-3H and 4A-4D).

FIGS. 6A-6C is the DNA and encoded protein sequences [SEQ ID NOS: 6 and 7] for the CMV promoter and the extracellular domain of CTLA-4 in the vector CTLA4-Fcαtplink. The sequence 5′ to base 514 and 3′ of the Eag I site is the same as for CD80Fcαtplink.

FIG. 7 is a profile for chromatography of CD80-Igαtp on a Superdex 200 column. The first peak eluting at about 45 min is the hexameric protein complex while the second peak migrates at the position observed for monomeric CD80-Ig. The inset shows a coomassie stained pattern for the purified CD80-Igαtp protein on SDS/PAGE under reducing (R) and nonreducing (NR) conditions.

FIG. 8 is a chart showing equilibrium sedimentation (main panel) and sedimentation velocity (inset) analytical centrifugation of the CD80-Igαtp protein with a modeled fit to a hexamer/(hexamer)₂ equilibrium. The upper graph shows the residuals for the equilibrium sedimentation centrifugation.

FIG. 9 is a line graph illustrating the binding of biotinylated CD80-Igαtp (labeled B7-FcA) to CD28-Ig immobilized at three different concentrations in an ELISA format. Binding was inhibited by the mAb CD28.1 or by CTLA4-Ig.

FIG. 10 is a line graph illustrating the binding of biotinylated CD80-Igαtp, CD86-Igαtp, and CD80-Ig compared to immobilized CD28-Ig in an ELISA format.

FIG. 11 is a line graph illustrating the binding of biotinylated CD80-Igαtp, CD86-Igαtp, and CD80-Ig compared to immobilized CTLA4-Ig in an ELISA format.

FIG. 12 is a line graph illustrating the competition of biotinylated CD80-Igαtp binding to immobilized CD28-Ig (coated at 200 mg/ml) by CD80-Igαtp itself, CD80-Ig, CTLA4-Ig, and CD28.2 MAb.

FIG. 13A is a line graph illustrating the binding of CD80-Igαtp to wild-type and mutant immobilized CD28-muIg2a proteins.

FIG. 13B is a line graph illustrating the binding of CD86-Igαtp to wild-type and mutant immobilized CD28-muIg2a proteins.

FIG. 13C is a line graph illustrating the binding of rabbit polyclonal antisera to wild-type and mutant immobilized CD28-muIg2a proteins.

FIG. 14 is a chart illustrating sequentially the binding of CD80-Ig and CD80-Igαtp to CD28-Ig immobilized on a biosensor chip as measured by surface plasmon resonance.

FIG. 15 is a chart illustrating the binding of CD80-Igαtp and CD86-Igαtp to CD28-Ig immobilized on a biosensor chip as measured by surface plasmon resonance.

FIGS. 16A and 16B are line graphs illustrating the binding of CD80-Igαtp and CD86-Igαtp, respectively, to cells expressing human CD28 on their surface in the presence or absence of a CD28 monoclonal antibody that inhibits this interaction.

FIG. 17 is a bar chart illustrating the level of IL-2 production by PCD28.1 cells treated with monomeric and hexameric CD80 (labeled B7.1-Ig and B7.1-IgA, respectively) and CD86 (labeled B7.2-Ig and B7.2-IgA, respectively) Ig fusion proteins. The proteins were used (1) alone in solution, (2) alone immobilized through goat anti-human antibody (GAH), or (3) immobilized in combination with immobilized CD3 mAb. Controls were GAH alone, or with CD3 mAb, and the CD28 IgM mAb 248.23.2. IL-2 levels were determined by CTLL-2 bioassay using known amounts of IL-2 as a standard (inset).

FIG. 18 is a bar chart illustrating the level of IL-2 production by DC27.CD28wt cells treated as described in FIG. 17.

FIG. 19 is a bar chart illustrating IL-2 promoter activity in PCD28.1 cells stimulated as described in FIG. 17. IL-2 promoter activity was measured by induction of β-galactosidase activity which serves as a reporter gene under the control of an IL-2 promoter.

FIGS. 20A and 20B are bar graphs respectively showing the induction of the IL-2 promoter, and IL-2 production by CD28 expressing cells incubated with CD80-Igαtp, CD86-Igαtp, or CD80-Ig.

FIG. 20C is a bar graph showing the levels of IL-2 production induced with soluble CD80-Igαtp and CD86-Igαtp in comparison to that induced by immobilized antibody to CD3.

FIG. 21 is a bar chart illustrating inhibition of biotinylated CD80-Igαtp binding to immobilized CD28-Ig by individual compounds in the BM-34 test set. The percent inhibition range is plotted against the number of compounds showing that range of inhibition.

FIG. 22 is a profile for Superose 6 chromatography of the chimeric derivative of the Epo receptor antibody 1C8 (here labeled “anti-EPOr-IgG₁”) and the αtp construct of the same antibody (labeled “anti-EPOr-IgG₁αtp”) with binding activity to an immobilized EPOr-Ig protein shown in the inset.

### DETAILED DESCRIPTION OF THE INVENTION

The invention provides an hexameric fusion protein useful in therapeutic and immunogenic compositions. The hexameric fusion protein of the invention is particularly well suited for applications for which the binding protein from which it is derived is unsatisfactory because of low binding affinity/avidity and for other applications where multivalency is desired. These applications include diagnostics, binding assays, screening assays and cellular responses based on receptor cross-linking. Also provided are compositions and methods for production and purification of these fusion proteins.

The invention further provides methods of producing stable hexameric fusion proteins, by providing a selected binding protein with an IgA tailpiece (αtp) or a functional equivalent thereof. The inventors have found that addition of the αtp from the natively monomeric or dimeric IgA, surprisingly, provides the resulting fusion protein with the ability to form stable hexamers.

I. Fusion Proteins

As used herein, a hexameric fusion protein of the invention contains a dimeric binding protein which has been provided at its carboxy terminus with a tailpiece (αtp) characterized by having the activity of the tailpiece from the C-terminus of the heavy chain of an IgA antibody. This tailpiece, when attached to each monomer of the dimeric binding protein, provides the resulting fusion protein with the ability to form stable hexamers, i.e., the hexameric fusion proteins of the invention do not undergo any appreciable dissociation in solution (e.g., phosphate buffered saline) at room temperature.

In a particularly preferred embodiment, the fusion proteins of the invention are homo-hexamers. However, where desired, hetero-hexamers comprising two different fusion proteins may be constructed.

The binding proteins useful in the invention include full-length proteins and fragments thereof which are characterized by the binding ability of the full-length protein, i.e., the fragment which has the ability to bind to the counter-receptors or other ligands of the selected binding protein. Such binding proteins may be derived from a protein or protein complex which natively dimerizes for biological activity, or may be genetically engineered as described herein. Examples of suitable natively dimeric binding proteins are those with carboxyl termini situated such that addition of the αtp to the carboxyl terminus of each polypeptide chain, with or without a linker, allows juxtaposition of the αtp chains. One of skill may readily select such native dimeric proteins or dimeric protein complexes, which include, for example, IgG, IgD, or IgE antibodies, Fab fragments, Fab₂ fragments, Ig-Fc fragments, Ig fusion proteins, and the extracellular domains of cell surface proteins such as the α/β chain of a T cell receptor, CD28 and CTLA4, CD8 α/β hetorodimers and α/α homodimers, and the α/β chain of integrin proteins and various cytokine receptors (e.g., IL3, IL5, etc.). These binding proteins are available from a variety of commercial and academic sources. Alternatively, these sequences may be chemically synthesized.

As discussed above, a selected binding protein may be engineered to be dimeric. For example, a protein fragment comprising a binding domain of a selected monomeric binding protein may be attached to an Ig-Fc fragment which forms dimers. Desirably, the binding protein is selected from surface glycoproteins from the immunoglobulin supergene family and their ligands. For example, in a currently preferred embodiment, the binding protein is selected from CTLA-4 (whose extracellular domain can be expressed as a monomer or dimer) and its counter-receptors CD80 and CD86. However, other proteins, including other binding proteins, are known to those of skill in the art and may be used in the construction of a hexameric fusion protein of the invention. Although a currently preferred embodiment of this invention provides hexameric immunoglobulin fusion proteins, which are exemplified herein, this invention is not so limited. For example, a binding protein may be genetically modified to alter its activity. For example, engineered, mutant forms of IL4 have been described that retain high affinity for its receptor but lack normal agonist activity and serve as antagonists of IL-4 mediated function [see, e.g., N. Kruse et al, EMBO J., 11:3237-3244 (1992) and WO96/04388 (Feb. 15, 1996)]. Such a mutant would be useful in a hexameric IL4-Ig fusion protein according to the invention, serving as an antagonist of IL4 function.

The protein fragment used to construct a dimeric binding protein contains at least a fragment of the extracellular domain of the selected binding protein. For functional binding activity, this extracellular fragment preferably contains the sequences required for binding, which can be readily determined by one of skill in the art. In a preferred embodiment, which makes use of a eukaryotic production system, the protein fragment also contains an export leader sequence which is native to the binding protein selected. However, other export leader sequences which are capable of exporting the protein may be substituted by one of skill in the art. In one exemplary embodiment, where the target is CD28, the protein fragment is the native leader and extracellular domain from CD80 or CD86. The fragments can be obtained from proteins such as CD80 [P. S. Linsley et al., J. Exp. Med., 173(3):721-730 (1991); Truneh et al., Mol. Immunol., 33(3):321-334 (1996); J. E. Ellis et al., J. Immunol., 56:2700-2709 (1996)], and CD86 [P. S. Linsley et al., Immunity, 1:793-801 (1994); J. E. Ellis et al., cited above; P. S. Linsley et al., J. Exp. Med., 174:561-569 (1991)]. In another embodiment, where the target is CD80 or CD86, the protein fragment is the native leader and extracellular domain from CTLA-4 or CD28.

The Fc fragment used in the construction of the hexameric fusion protein may be from any antibody subclass, except IgA. Thus, the Fc fragment may be derived from the IgG, IgD, or IgE subclass. When the Fc fragment is derived from an IgG antibody, any of the human isotypes, i.e., IgG₁, IgG₂, IgG₃, and IgG₄, may be selected. Further, the parental IgG antibody may be mutated to reduce binding to complement or Ig-Fc receptors [see, e.g., A. R. Duncan et al., Nature, 332:563-564 (1988); A. R. Duncan and G. Winter, Nature, 332:738 (1988); M. -L. Alegre et al., J. Immunol., 148:3461-3468 (1992); M-H Tao et al., J. Exp. Med., 178:661-667 (1993); V. Xu et la, J. Biol. Chem., 269:3469-3474 (1994)]. When the Ig-Fc fragment is derived from IgM, it desirably contains the hinge/CH2/CH3/CH4 sequence, but not the naturally occuring 18 amino acid tailpiece (μtp).

Optionally, the C-terminal end of the IgG₁ CH3 domain of the Fc fragment may be modified by conventional techniques to contain a restriction enzyme site for convenient cloning of the tailpiece segments (i.e., the peptide of the invention). Such modifications are described in more detail in the examples below, and are well known to those of skill in the art.

The peptide used to construct the fusion protein of the invention is derived from tailpiece located at the C-terminus of the heavy chain of an IgA antibody. In a preferred embodiment, this peptide is 18 residues in length and is the αtp segment of the human IgA1 heavy chain or a functional equivalent thereof. One particularly suitable peptide is: PTHVNVSVVMAEVDGTCY [SEQ ID NO: 3]. If desired, this peptide may be modified to remove the glycosylation site by changing 1 or 2 amino acids at residues 5-7 (NVS). For example, the N (asparagine) may be to changed to Q (glutamine) and/or the S (serine) may be changed to A (alanine). Additionally, up to about 4 amino acid residues of the human IgA CH3 domain may be retained, Alternatively, functional equivalents of the human IgA1 αtp may be selected. Suitable functional equivalents include, for example, gorilla IgG1, human IgA2, rabbit IgA, and mouse IgA. Such functional equivalents may also be modified by removal of glycosylation sites. As described herein, this peptide is linked, directly or indirectly, to the binding protein (e.g., the Ig-Fc fragment) and provides the fusion protein of the invention with the ability to assemble into a stable hexamer.

The fusion protein may contain a linker sequence. Optionally, such a linker may be located between the binding protein (e.g., the Ig-Fc fragment) and the αtp peptide. This linker is preferably an amino acid sequence between about 1 and 20 amino acid residues, and more preferably between about 1 and 12 amino acid residues, in length. Other appropriate or desired linkers may be readily selected by one of skill in the art. Although currently less desired, one of skill in the art may substitute other linkers for the preferred amino acid sequence linkers described above.

Three currently preferred embodiments of the fusion proteins of the invention are described herein, CD80-Igαtp, CD86-Igαtp and CTLA4-Igαtp. These proteins are composed of the native leader and extracellular domains of the CD80 (B7.1), the CD86 (B7.2, B70), and the CTLA4 surface glycoproteins, respectively, linked to the hinge/CH2/CH3 region of the heavy chain of human IgG₁ (Fc fragment) and terminating in a short tail piece segment from human IgA1 (αtp). Another example of a hexameric protein of the invention is an IgG antibody, where the αtp is joined directly to the carboxy terminus of the heavy chain and a light chain is paired with this heavy chain. The αtp hexameric antibody and Ig fusion proteins of the invention are advantageous over IgM antibodies and IgM fusion proteins in that the hexamers of the invention are readily purified on commercially available chromatography supports and are more efficiently expressed.

These constructs may be made using known techniques. A detailed description of the construction of these exemplary fusion proteins of the invention is provided in the examples below.

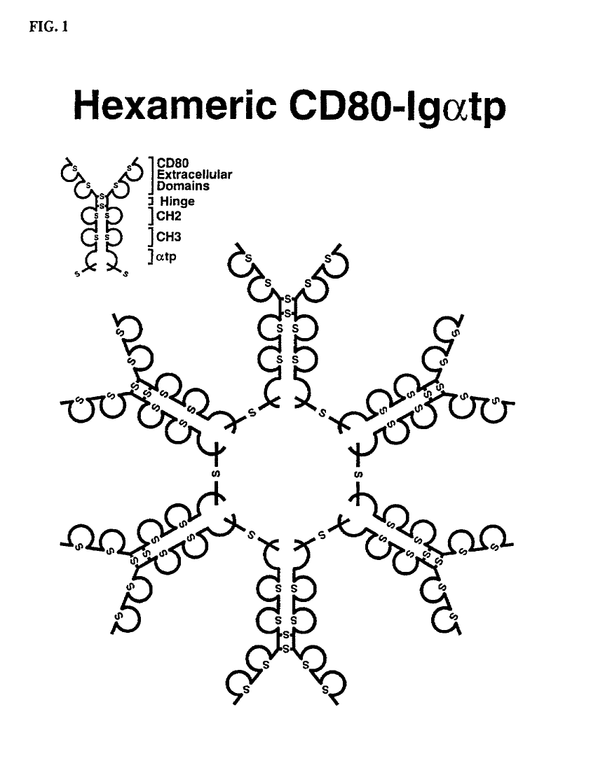

Briefly, each chain of a dimeric binding protein is selected or constructed. For example, one preferred binding protein is a recombinant immunoglobulin containing the native leader and extracellular domain fused to an Ig-Fc fragment from the selected human IgG antibody. The αtp is added, optionally by introducing a convenient restriction endonuclease site near the C-terminus of the binding protein (e.g., an Fc region) using silent mutations of the coding sequence and then cloning a synthetic oligonucleotide into this site that encodes the tailpiece segment. The tailpiece segment is matched to that of the human α-1 chain. The tailpiece provides the fusion protein with the ability to form hexamers and the resulting construct is the hexameric fusion protein of the invention. A schematic representation of the predicted hexamer for an exemplary fusion construct of the invention, CD80-Igαtp, is shown in FIG. 1.

Preferably, the fusion proteins of the invention are produced using recombinant techniques. Desirably, the nucleic acid sequences may be fused and the fusion protein expressed in vitro in a suitable host cell. Alternatively, the fusion proteins of the invention are produced by separately expressing, or co-expressing the nucleic acid sequences encoding the protein fragments and αtp fragment of the invention and fusing the expressed products. Suitably, the resulting fusion protein forms hexamers. These production techniques are discussed in more detail below.

II. Polynucleotide Sequences, Expression and Purification

The present invention further encompasses polynucleotide sequences encoding the fusion proteins of the invention. In addition to the DNA coding strand, the nucleic acid sequences of the invention include the DNA (including complementary DNA) sequence representing the non-coding strand and the messenger RNA sequence. Variants of these nucleic acids of the invention include variations due to the degeneracy of the genetic code and are encompassed by this invention. Such variants may be readily identified and/or constructed by one of skill in the art. Further, the polynucleotide sequences may be modified by adding readily assayable tags to facilitate quantitation, where desirable.

To produce recombinant fusion proteins of this invention, the DNA sequences of the invention are inserted into a suitable expression system, preferably a eukaryotic system. Desirably, a recombinant vector is constructed in which the polynucleotide sequence encoding at least one chain of the fusion protein (i.e., the binding protein/αtp) is operably linked to a heterologous expression control sequence permitting expression of the fusion protein of the invention. Numerous types of appropriate expression vectors and host cell systems are known in the art for expression, including, e.g., mammalian, yeast, bacterial, fungal, drosophila, and baculovirus expression.

The transformation of one or more of these vectors into appropriate host cells results in expression of the fusion proteins of the invention. Other appropriate expression vectors, of which numerous types are known in the art, can also be used for this purpose.

Such production methods permit assembly of the hexameric fusion protein of the invention by the host cell. Typically, such methods will provide a homo-hexameric fusion protein. However, in another embodiment, hexameric fusion proteins of mixed specificity may be produced by co-expression of different fusion proteins (i.e., binding protein/αtp). For example, two fusion proteins recognizing non-competing sites on the same molecule can be co-expressed resulting in hexamers that can bind to two sites on the same molecule, resulting in higher binding avidity than for each fusion protein alone or as a homogenous hexamer. Alternatively, the two fusion proteins can bind to two distinct molecules presented on the same, or different surfaces (e.g., expressed on the same or different cells).

Suitable host cells or cell lines for transfection by this method include mammalian cells, such as Human 293 cells, Chinese hamster ovary cells (CHO), the monkey COS-1 cell line, murine L cells or murine 3T3 cells derived from Swiss, Balb-c or NIH mice. Suitable mammalian host cells and methods for transformation, culture, amplification, screening, and product production and purification are known in the art. [See, e.g., Gething and Sambrook, Nature, 293:620-625 (1981), or alternatively, Kaufman et al., Mol. Cell. Biol., 5(7):1750-1759 (1985) or Howley et al., U.S. Pat. No. 4,419,446]. Another suitable mammalian cell line is the CV-1 cell line.

Other host cells include insect cells, such as Spodoptera frugipedera (Sf9) cells. Methods for the construction and transformation of such host cells are well-known, [See, e.g. Miller et al., Genetic Engineering, 8:277-298 (Plenum Press 1986) and references cited therein].

Although less preferred, also useful as host cells for the vectors of the present invention are bacterial cells. For example, the various strains of E. coli (e.g., HB101, MC1061) are well-known as host cells in the field of biotechnology. Various strains of B. subtilis, Pseudomonas, other bacilli and the like may also be employed in this method.

Many strains of yeast cells known to those skilled in the art are also available as host cells for expression of the proteins of the present invention. Other fungal cells may also be employed as expression systems.

Thus, the present invention provides a method for producing a fusion protein of the invention which involves transforming a host cell, preferably a eukaryote, with at least one expression vector containing a recombinant polynucleotide encoding a fusion protein under the control of a transcriptional regulatory sequence, e.g., by conventional means such as transfection or electroporation. The transformed host cell is then cultured under suitable conditions that allow expression of the fusion protein. The expressed and assembled fusion protein is then recovered, isolated, and purified from the culture medium by appropriate means known to one of skill in the art. In a preferred embodiment, the fusion proteins are assembled by the host cell following co-production of one or more of the fusion proteins of the invention. Alternatively, the hexameric fusion protein may be assembled following recovery from the host cell.

Advantageously, the fusion proteins of the invention can be readily purified using conventional techniques. For example, hexameric Ig fusion proteins of the invention may be readily purified on high affinity, high capacity supports based on protein A and protein G. Such resins are commercially available [Pharmacia Inc.; Bioprocessing Ltd.].

Although less preferred, the hexameric fusion protein may be produced in insoluble form. For example, the proteins may be isolated following cell lysis in soluble form, or extracted in guanidine chloride.

III. Pharmaceutical Compositions and Methods of Use Thereof

The fusion proteins of this invention or DNA sequences encoding them may be formulated into pharmaceutical compositions and administered using a therapeutic or immunogenic regimen compatible with the particular formulation. Pharmaceutical compositions within the scope of the present invention include compositions containing a protein of the invention in an effective amount to have the desired physiological effect.

Suitable formulations for parenteral administration include aqueous solutions of the active compounds in water-soluble or water-dispersible form, e.g., saline. Alternatively, suspensions of the active compounds may be administered in suitable conventional lipophilic carriers or in liposomes. In still another alternative, adjuvants may be desired, particularly where the composition is to be used as an immunogen.

The compositions may be supplemented by active pharmaceutical ingredients, where desired. Optional antibacterial, antiseptic, and antioxidant agents in the compositions can perform their ordinary functions. The pharmaceutical compositions of the invention may further contain any of a number of suitable viscosity enhancers, stabilizers, excipients and auxiliaries which facilitate processing of the active compounds into preparations that can be used pharmaceutically. Preferably, these preparations, as well as those preparations discussed below, are designed for parenteral administration. However, compositions designed for oral or rectal administration are also considered to fall within the scope of the present invention.

As used herein, the terms “suitable amount” or “effective amount” means an amount which is effective to treat or prevent the conditions referred to below. A preferred dose of a pharmaceutical composition containing a fusion protein of this invention is generally effective above about 0.1 mg fusion protein of the invention per kg of body weight (mg/kg), and preferably from about 1 mg/kg to about 100 mg/kg. These doses may be administered with a frequency necessary to achieve and maintain satisfactory fusion protein levels. Although a preferred range has been described above, determination of the effective amounts for treatment or prophylaxis of a particular condition may be determined by those of skill in the art.

Particularly, pharmaceutical compositions containing the hexameric antibody/αtp fusion proteins of the invention are useful as antagonists for the 7 transmembrane (7 TMR) class of cell surface receptors, since such receptors are often arrayed in many copies on cell surfaces and the aggregation of such receptors does not lead to intracelluar signalling (agonism) as can occur for many other types of cell surface receptors. For example, administration of a pharmaceutical compositions containing a hexameric antibody/αtp fusion protein of the invention blockades chemokine receptors, a subfamily of the 7 TMR, and inhibits chemotaxis and activation of target cells such as eosinophils. A second example is CTLA4-Igαtp. CTLA4-Ig is a potent inhibitor of CD80 and CD86 driven stimulation of T-cells through their interaction with CD28. In animal models, CTLA4-Ig has shown benefit in several autoimmune diseases and transplantation.

Since the CD80 and CD86 antigens recognized by CTLA4-Ig are arrayed in many copies on the cell surface, an αtp hexameric form of CTLA4-Ig may provide a more potent antagonist than the standard Ig fusion protein. In another embodiment, a pharmaceutical composition of the invention containing Igαtp fusion proteins of the invention may be used for removal of complement components or components of the blood coagulation cascade to retard clotting.

In one aspect, the invention provides a method for antagonizing cell surface CD80- and CD86-mediated stimulation of CD28 positive cells by administering to the cells a hexameric fusion protein CTLA4-Igαtp. This may be performed in vivo, by administering a pharmaceutical composition containing this hexameric fusion protein. In another aspect, the invention provides a method for stimulating (agonist activity) CD28+ T cells by administering the CD80- or CD86hexameric fusion protein to the cells in culture resulting in stimulation of IL-2 production from these cells. These proteins may be used alone, or in combination with other stimulators of T-cells (e.g., antibodies directed against the T cell receptor-CD3 complex.)

In another embodiment, the compositions of the invention containing Ig-Fc-containing fusion proteins are useful for in vivo clearance of soluble ligands, in view of the fact that hexamerization of the Fc domain enhances interaction with complement components and Fc receptors. Thus, ligands bound to the hexameric fusion protein of the invention are efficiently cleared from circulation.

The hexameric fusion proteins of the invention can also serve as agonists, particularly in situations where aggregation can induce a desired response. For example, aggregation is essential for signal transduction through many cell surface receptors—either as a consequence of multivalent presentation of the receptor ligand (eg., a counter receptor on a the surface of a second cell) or through changes induced upon ligand binding, or both. An example of signalling through a cell surface receptor induced by cross-linking through recognition of its counter-receptor on a second cell is CD28 recognition by CD80 or CD86.

Thus, the invention further provides a method for stimulating CD28 positive cells by administering to CD28 positive cells CD80-Igαtp and/or CD86-Igαtp. Examples of soluble ligands inducing signal transduction through binding to their receptors are EGF and growth hormone and both result in receptor dimerization. For these receptors, dimerization induced through antibody binding also can lead to activation [Schreiber et al., Proc. Natl. Acad. Sci. USA, 78:7535 (1981), Fuh et al., Science, 256:1677 (1992)]. Hexameric antibodies against such receptors or hexameric ligand-Ig fusion proteins for these receptors are expected to be more efficient stimulators than the standard dimeric antibodies or ligand Ig fusion proteins. For example, the pharmaceutical compositions containing the hexameric antibodies or cytokine-Ig fusion proteins of the invention are useful in inducing signal transduction in receptors for hematopoietic cytokines, such as erythropoietin, thymopoietin and growth stimulatory factor.

Also provided is a method for suppressing CTLA-4 positive cells by administering CD80-Igαtp and/or CD86-Igαtp to CTLA4 positive cells. This may be performed in vivo, by administration of a pharmaceutical composition containing the hexameric proteins. Alternatively, the hexameric proteins are added to CTLA4 positive T-cells in culture resulting in inhibition of IL-2 production from these cells.

In yet another aspect, hexameric Ig-fusion proteins of the invention can also serve as enhanced immunogens for the fused protein fragment due to efficient, receptor-mediated updake for antigen processing and presentation or efficient interaction with proteins of the complement system. Enhanced immunogenicity is desirable for the efficient generation of polygonal and monoclonal antibodies and for therapeutic vaccination. Thus, the invention further provides a method of immunizing using the pharmaceutical composition of the invention.

IV. Assays

The hexameric fusion proteins of the invention are useful in in vitro assays for measuring the binding of the fusion protein to a selected ligand and for identifying the native or synthetic ligand for the binding proteins. Such a ligand includes the native ligand or counter-receptor to the binding protein from which the hexameric fusion protein is derived. For example, where the fusion protein is derived from CD80 or CD86, the ligand may be CD28 or CTLA-4. Alternatively, the ligand may be a derivative of the native counter-receptor, a peptide, peptide-like compound, or a chemical compound which interacts with the fusion protein.

The hexameric fusion proteins may be used for in vivo assays, including, for example imaging. See, e.g., S. M. Larson et al., Acta Oncologica, 32(7-8):709-715 (1993); R. DeJager et al., Seminars in Nuclear Medicine, 23(2):165-179 (Apr. 1993).

Alternatively, a fusion protein of the invention may be used to screen for new ligands. The use of the fusion proteins of this invention in such an assay is particularly well suited for identifying cell surface or multivalent ligands.

Suitable assay methods may be readily determined by one of skill in the art. For example, an ELISA format may be utilized in which the selected ligand is immobilized, directly or indirectly (e.g., via an anti-ligand antibody) to a suitable surface.

Where desired, and depending on the assay selected, the hexameric fusion protein may be immobilized on a suitable surface. Such immobilization surfaces are well known. For example, a wettable inert bead may be used in order to facilitate multivalent interaction with the hexameric fusion proteins of the invention.

Further, the methods of the invention are readily adaptable to combinatorial technology, where multiple molecules are contained on an immobilized support system. Thus, the fusion proteins of the invention permit screening of chemical compound and peptide based libraries where these agents are presented in a multivalent format compatible with more than one subunit of the hexamer. Monomeric interactions of this type are routinely in the mM range and thus may not be readily detected with monomeric proteins. Advantageously, the avidity of the hexameric fusion proteins of the invention permit direct binding.

Typically, the surface containing the immobilized ligand is permitted to come into contact with a solution containing the fusion protein and binding is measured using an appropriate detection system. Suitable detection systems include the streptavidin horse-radish peroxidase conjugate, direct conjugation by a tag, e.g., fluorescein. Other systems are well known to those of skill in the art. This invention is not limited by the detection system used.

The assay methods described herein are also useful in screening for inhibition of the interaction between a hexameric fusion protein of the invention (and thus, the binding protein from which it is derived) and its ligand(s). For example, one may screen for inhibitors of CD80 and CD86 binding to CD28 and CTLA-4. In a preferred method, a solution containing the suspected inhibitors is contacted with an immobilized recombinant CD28 or CTLA-4 protein substantially simultaneously with contacting the immobilized ligand with the solution containing the hexameric CD80- or CD86-Igαtp protein. The solution containing the inhibitors may be obtained from any appropriate source, including, for example, extracts of supernatants from culture of bioorganisms, extracts from organisms collected from natural sources, chemical compounds, and mixtures thereof. In another variation, the inhibitor solution may be added prior to or after addition of the CD80- or CD86-Igαtp proteins to the immobilized CD28 or CTLA-4 protein. Similar methods may be performed using other hexameric fusion proteins of the invention and their respective ligands.

The large size of the Igαtp fusion proteins is also advantageous for biophysical assay methods dependent on diffusion or rotation of the protein target in solution, such as for example, fluorescence polarization, fluorescence correlation spectroscopy and anisotropic analytical methods.

These examples illustrate the preferred methods for preparing and using the fusion proteins of the invention. These examples are illustrative only and do not limit the scope of the invention.

### EXAMPLE 1

### Production and Characterization of Exemplary αtp Ig Fusion Proteins

The following describes the production of CD80-Igαtp, CD86-Igαtp, and CTLA4 -Igαtp. Further, for comparison, a construct containing the human IgM tailpiece added to the C-terminus of CD80-Ig was also prepared. This construct, designated CD80-Igutp, differs in amino acid sequence from the αtp derivative as follows:

| | CH3 | Tailpiece | SEQ ID NO: |
|---|---|---|---|
| IgG1 | SLSPGK | (none) | 9 |
| μtp | SLSTGK | PTLYNVSLVMSDTAGTCY | 25 and 10 |
| αtp | SLSAGK | PTHVNVSVVMAEVDGTCY | 26 and 11 |

A. Construction of Recombinant Ig: Binding Protein Fragment/Fc Fusions

The pHbactCd28neo vector for expression of CD28 was previously described [D. Couez et al., Molecul. Immunol., 31(1):47-57 (1994)]. For expression of CD80, the coding sequence was cloned by PCR and inserted into a derivative [Dr. F. Letourneur, NIH] of pCDLSRα296 [Y. Takebe et al., Molecul. & Cell. Biol., 8(1):466-472 (1988)] as described [C. A. Fargeas et al., J. Exp. Med., 182:667-675 (1995)].

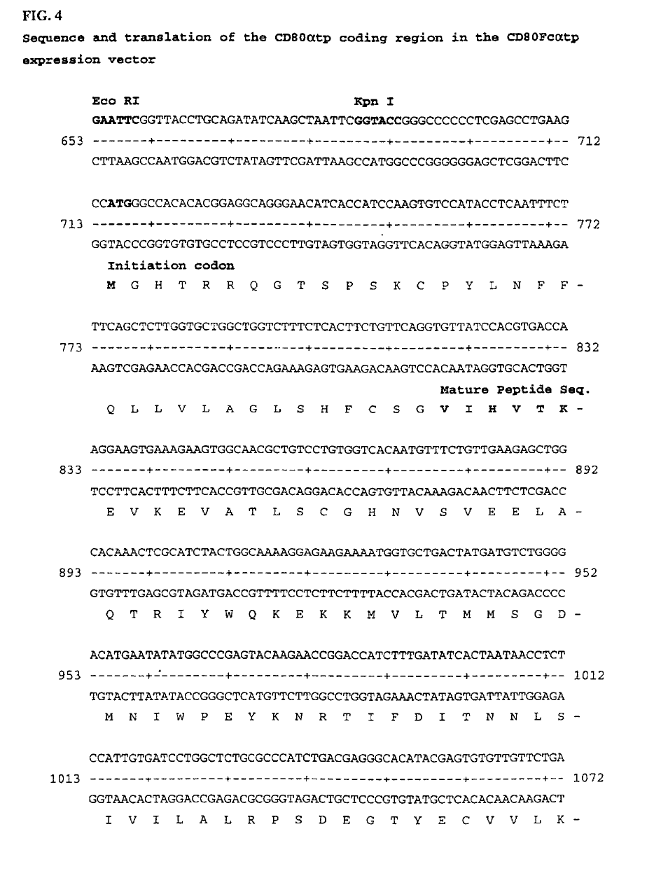

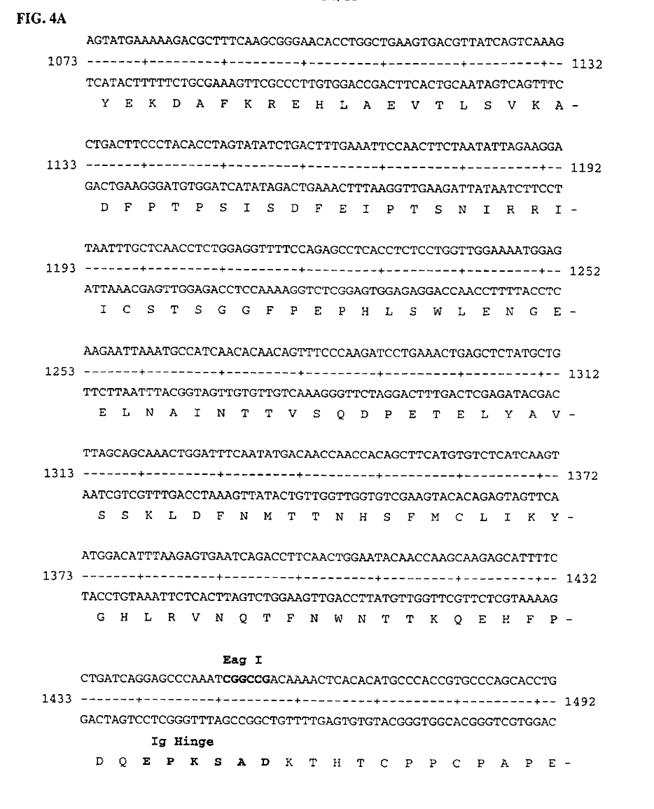

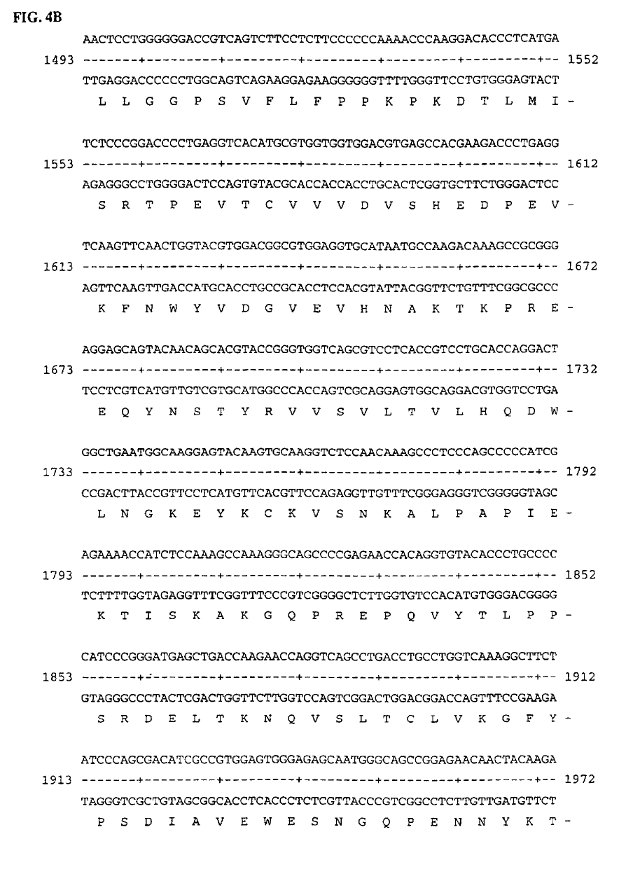

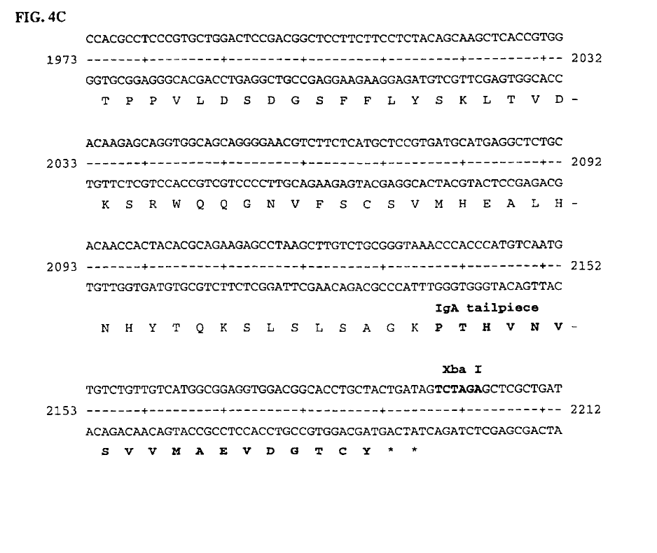

The vector COSFcLink [A. Truneh et al., Mol. Immunol., 33(3):321-334 (1996)] was constructed for expression of proteins C-terminally fused to a human IgG1 Fc region under the transcriptional control of the major late promoter of CMV. The dhfr cassette in this vector permits selection for gene amplification in response to methotrexate. The coding sequences for the native leader and extracellular domain peptide of CD28 and CD80 were grafted onto a human IgG1 heavy chain Fc region in the vector COSFcLink, beginning at the start of the hinge region, in a manner similar to that previously described for CD28 and CD80 [P. S. Linsley et al., J. Exp. Med., 174(3):561-569 (1991)]. The Fc region in this vector was derived from the human plasma leukemia cell line ARH-77 [ATCC CRL 1621] and contains a mutation of cysteine to alanine in the upper hinge region (SEQ ID NO: 27 EPKSA, where the mutation is underscored). The CD28 and CD80 sequences were cloned as KpnI-Eag I fragments by PCR from the vectors described above and inserted into the corresponding sites in COSFcLink. The resulting vectors are termed CD28FcLink and CD80FcLink, respectively. For CD28-Ig, the junction of receptor/Fc fragment (immunoglobulin junction) is SEQ ID NO: 12—GPSKP/EPKSA—and the mature processed N-terminal sequence is SEQ ID NO: 13 NKIL—. For CD80-Ig, the immunoglobulin junction is SEQ ID NO: 14—HFPDq/EPKSA—and the mature processed N-terminal sequence is VIHV—(FIGS. 4A-4D). The lower case “q” in CD80 represents the substitution of glutamine for the native asparagine.

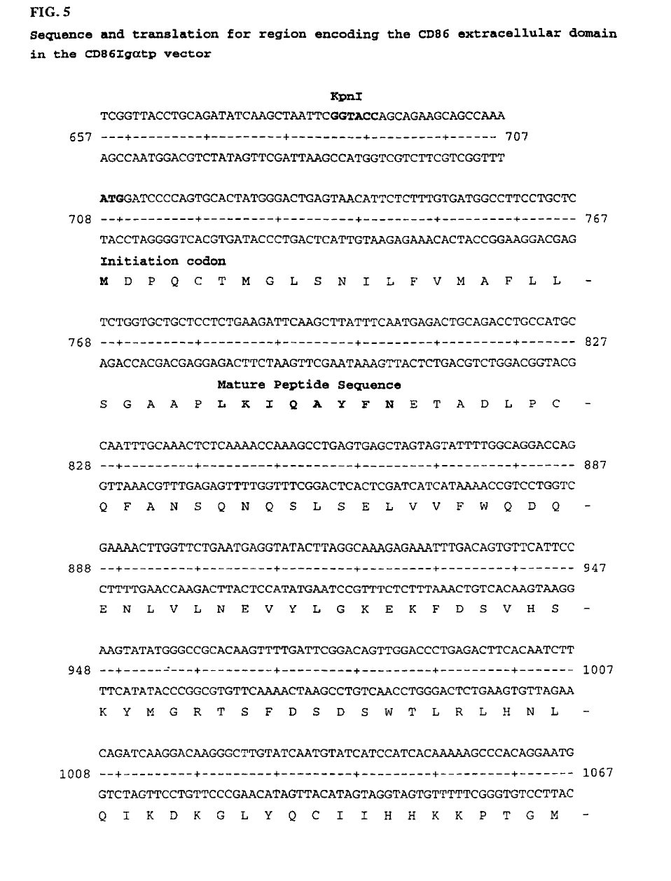

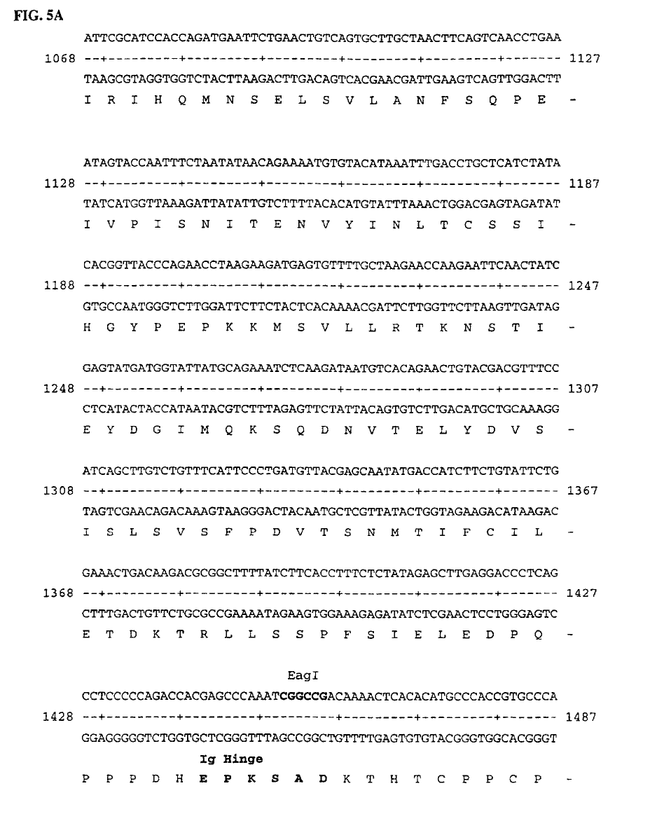

CD86-Ig, the corresponding binding protein/Fc construct for CD86 containing the native signal peptide of CD86 (B70) [M. Azuma et al., Nature, 366:76-79 (1993)], was constructed using methods essentially identical to those described above. The signal and extracellular sequences were PCR cloned from a plasmid containing the CD86 (B70) coding region that was obtained by reverse transcriptase/PCR cloning from human B-cell RNA based on the sequence described by M. Azuma et al. (above). Sequence analysis confirmed identity of this cloned CD86 (B70) region with that of Azuma et al. (above). The amino acid sequence at the junction to the Fc region is: SEQ ID NO: 16—PPPDHepksa—where capital and lower case letters indicate CD86 and Fc sequences respectively. The mature processed N-terminal sequence is SEQ ID NO: 17 LKIQ—(FIG. 5A-5B).

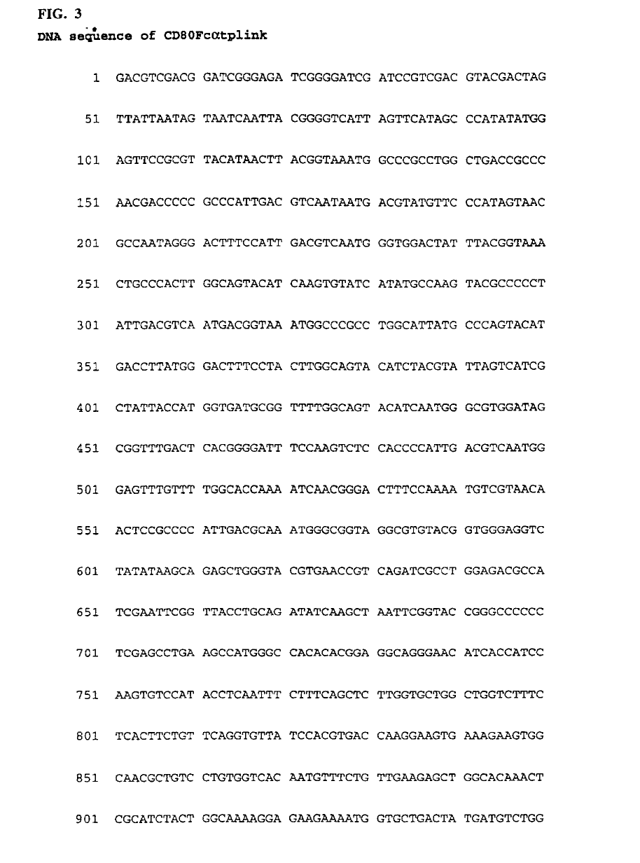

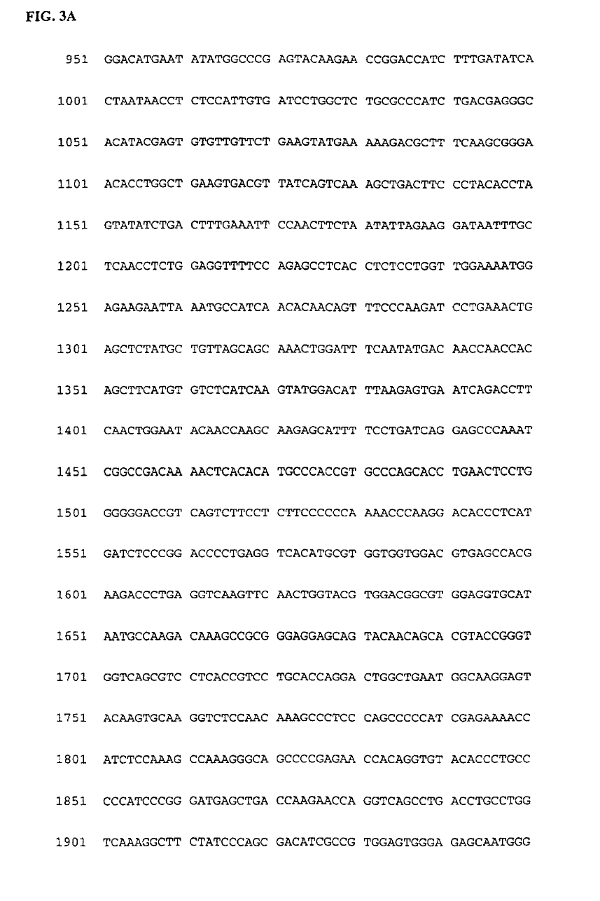

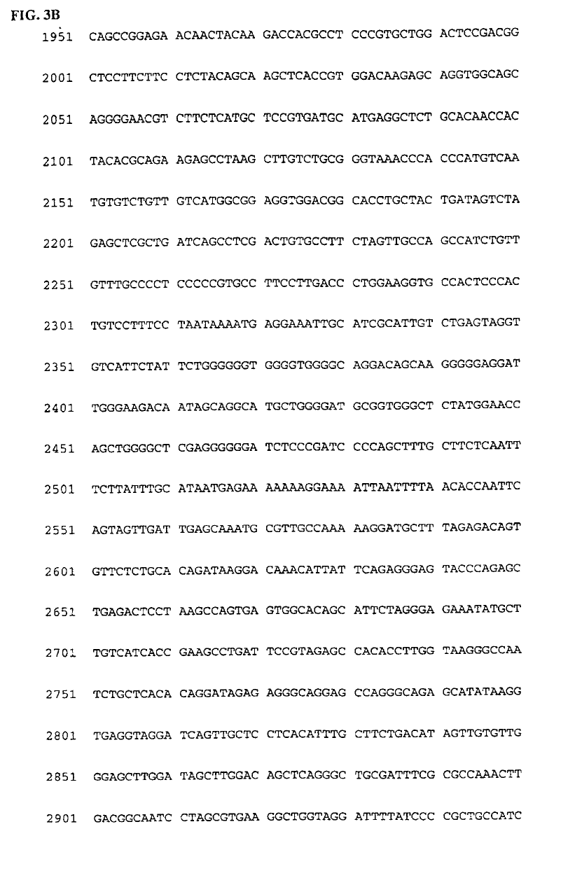

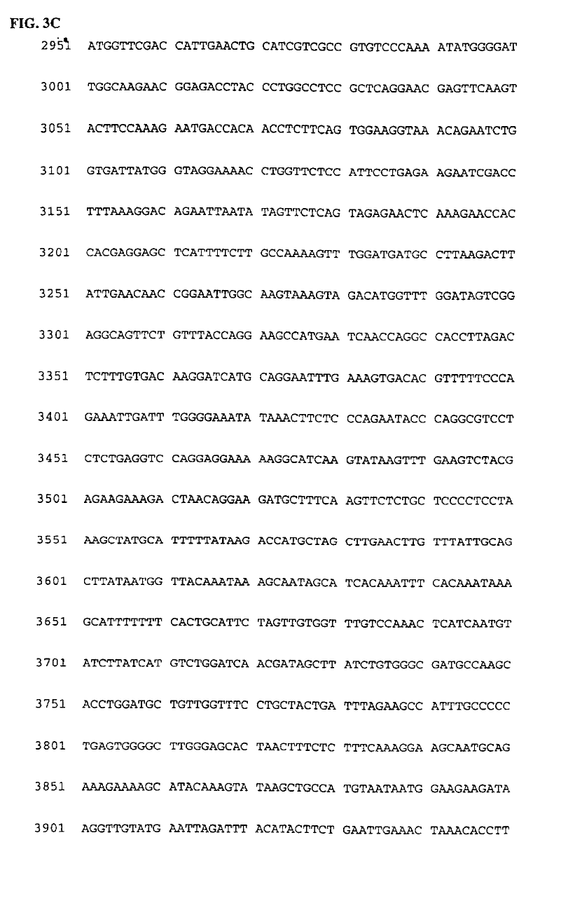

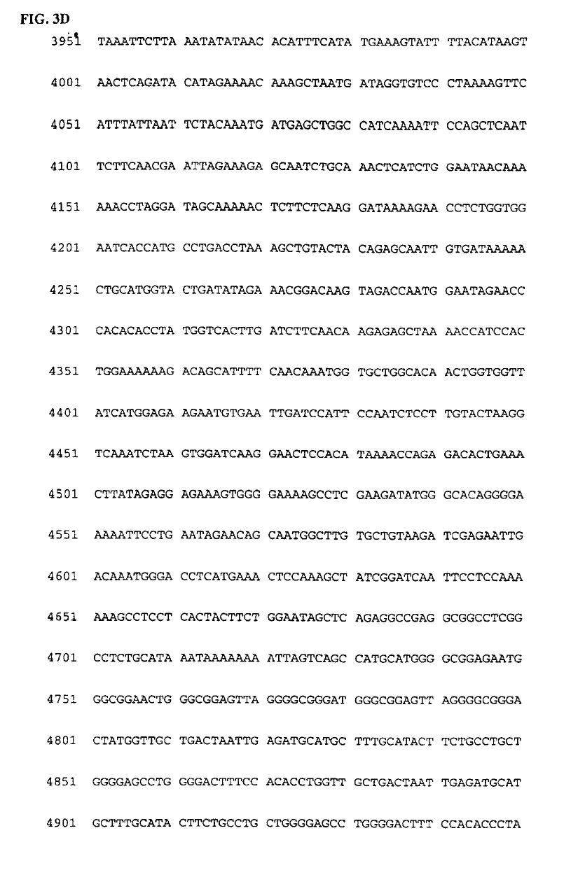

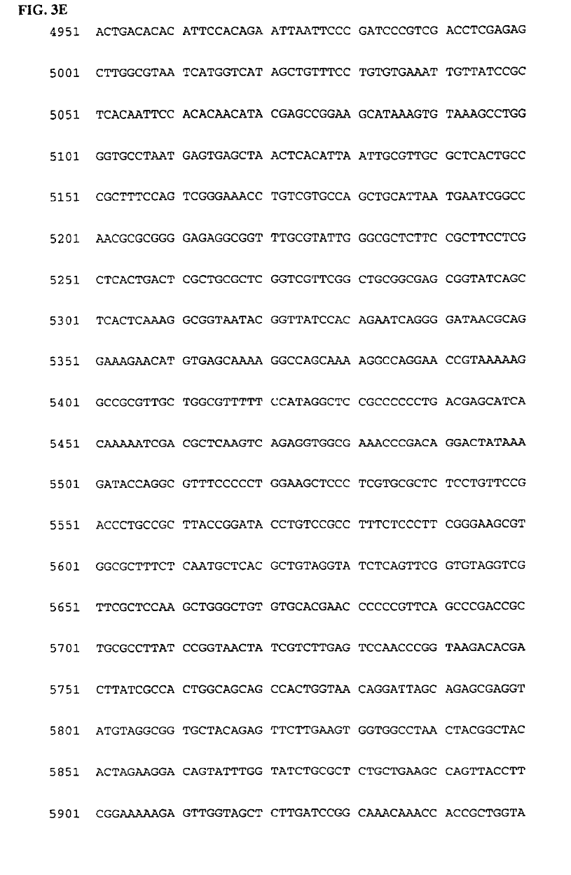

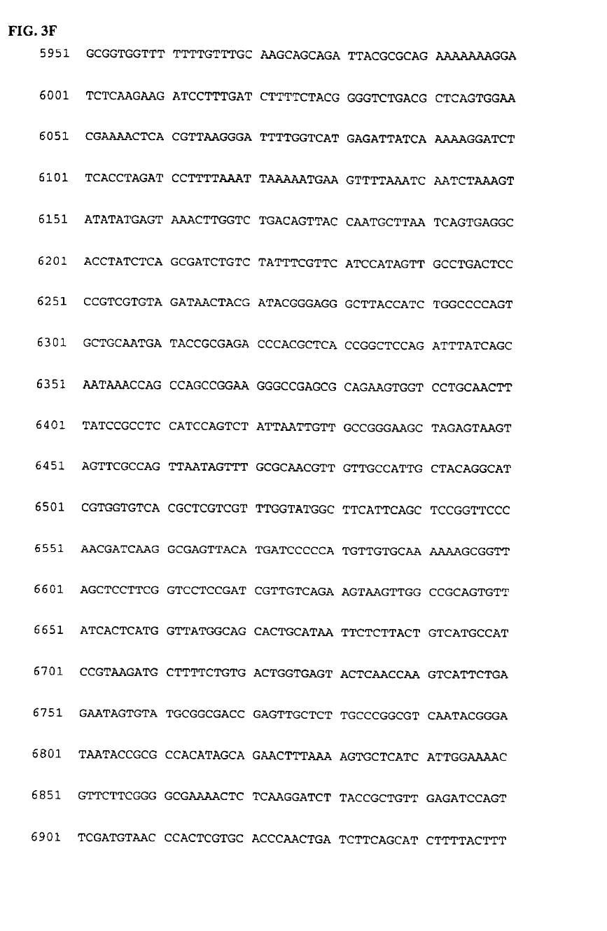

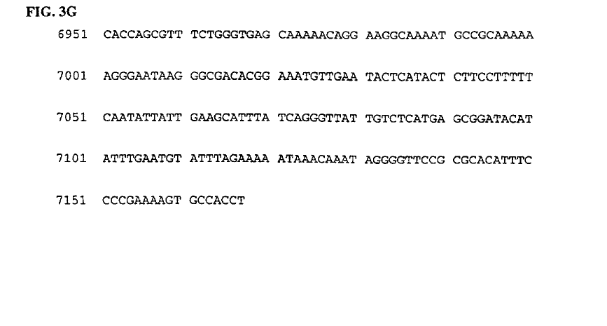

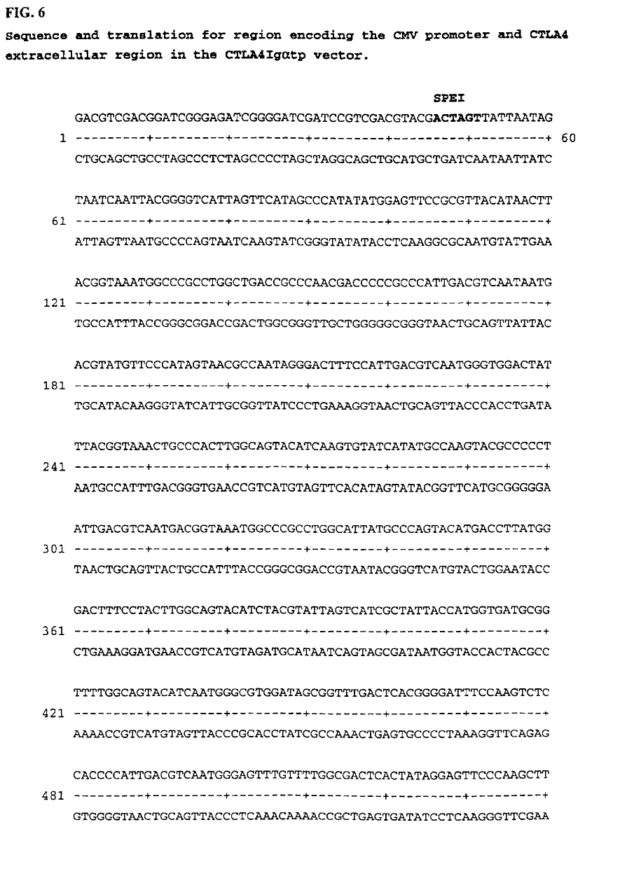

CTLA4-Ig, the corresponding binding protein/Fc construct for human CTLA4 containing the native signal peptide of CTLA4 [P. Dariavach et al., Eur J Immunol, 18: 1901-1905 (1988); Harper et al., J Immunol, 147: 1037-1044 (1991)] was constructed in a similar manner. HuC4.32, a pCDM8 plasmid containing the cDNA sequence for human CTLA4 (Harper et al., above) was provided by the laboratory of P. Golstein (Centre d'Immunologie INSERM-CNRS de Marseille-Luminy, 13288 Marseille Cedex 9, France). For PCR cloning of the extracellular domain, the 5′ primer was positioned in the pCDM8 vector. [Abberent cloning led to deletion of about 140 bp upstream of the EcoRI site relative to CD80Fclink (compare FIG. 6 with FIG. 3, below).] The amino acid sequence at the junction spanning the end of the CTLA4 extracellular domain and the hinge region is: SEQ ID NO: 18—EPCPDSDAepksa—where capital and lower case letters indicate CTLA4 and Fc sequences respectively and the underlined alanine residue indicates its substitution for phenylalanine in the native CTLA4 sequence. The mature processed N-terminal sequence is SEQ ID NO: 19 MHVA—(FIGS. 6A-6C).

Hexameric forms of the CTLA4, CD80 and CD86 recombinant Ig proteins were created by addition of a sequence encoding the 18 amino acid tail piece region of human IgA1 heavy chain to the C-terminus of the CH3 domain in the expression vectors described above. These methods are described in detail below.

B. Construction of Hexameric Fusion Proteins

For convenience, a Hind III site was introduced into the CH3 domain of CD80FcLink [spanning the 3rd base of the codon for Leu441 [EU numbering, E. A. Kabat et al., cited above] through the 2nd base of the codon for L443]. The Hind III site was introduced by standard PCR methods (eg., PCR Protocols: A Guide to Methods and Applications, Innis et al., eds, 1990) using the following oligonucleotides: 5′ oligo (positioned in the hinge region of the vector): SEQ ID NO: 20

```
                                                          EagI
                                          cccaaatcggccgacaaaact
3′ oligo (spanning the C-terminus of CH3): SEQ ID NO: 21
                            XbaI HindIII
tcagcgagctctagactacactcatttacccggagacaagcttaggctcttctgcgt
```

The PCR fragments were isolated by agarose gel electrophoresis and purified on Spin Bind columns (FMC Corp). The fragment was digested with Eag I and Xba I and cloned into similarly digested CD80FcLink vector and colonies were screened for the newly created Hind III site, yielding the vector CD80FcLink-Hd.

To introduce the αtp sequence, a synthetic oligonucleotide linker encoding this sequence was cloned between the newly created Hind III site and the Xba I site in CD80FcLink-Hd. The complementary oligonucleotides for the linker sequence were:

```
(5′) SEQ ID NO: 22 agcttgtctgcgggtaaacccacccatgtcaatgtgtctgttgtcatggc
          (Hind III adaptor)
ggaggtggacggcacctgctactgatagt
(5′) SEQ ID NO: 23 ctagactatcagtagcaggtgccgtccacctccgccatgacaacagac
          (Xba I adaptor)
acattgacatgggtgggtttacccgcagaca
```

5 mg of each linker was denatured at 70° C. for 10 minutes. The reactions were cooled to room temperature for 20 minutes. The concentration of linker was titrated from 50 to 5 ng using 1000 ng of gel purified CD80FcLink-Hd vector, digested with Hind III/Xba I. Several colonies from each ligation condition were screened for the presence of the αtp linker by PCR and confirmed by DNA sequencing.

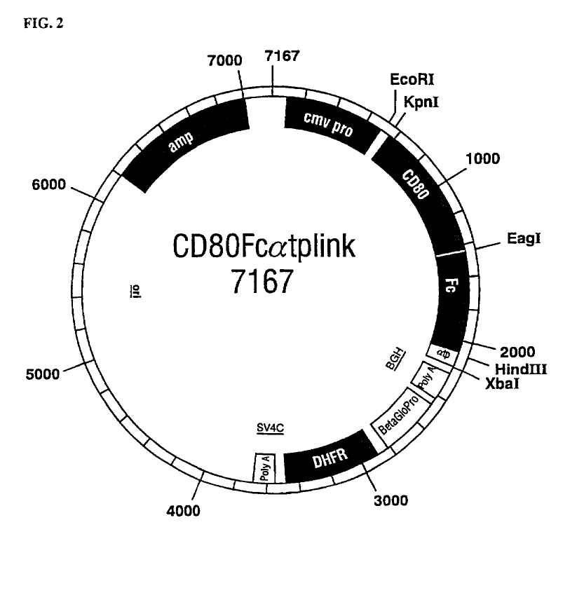

A schematic representation of the resulting vector, CD80Fcαtplink, is shown in FIG. 2 and the complete DNA sequence is given in FIGS. 3A-3H. The vector sequence may differ in some sites from the actual plasmid, but would be function. Introduction of the CD80-Igαtp coding region into other standard mammalian expression vectors (e.g., pBK-CMV from Stratagene. La Jolla, Calif.) will give suitable results and can be modified appropriately, e.g., by introduction of a dhfr gene, by one of skill in the art.

The vectors for expression of CD86-Igαtp as derived from the corresponding Ig expression vector by replacing the Fc coding region with the Fc-atp region from CD80-Igαtp. The Fc segment of CD86Fclink was excised by cleavage with Eag I (in the hinge region) and Xba I (following the C-terminus of CH3) and replaced with the corresponding fragment of CD80Fcatplink to give the expression vector CD86Fcatplink. The vector for expression of CTLA4-Igαtp was derived by replacing a SpeI-EagI fragment in CD80Fcatplink with the corresponding fragment from CTLA4Fclink to give the expression vector CTLA4Fcatplink. The SpeI site is at base 46 in the CMV promoter region. The sequences of the CD86 and CTLA4 constructs in the region differing from CD80Fcatplink are given in FIGS. 5 and 6.

By a similar approach a vector encoding CD28-Igαtp could be prepared starting the CD28Fclink vector described in part A above, or a similar construct encoding an altered version of the CD28 extracellular sequence.

C. Production and Purification

The CD28-Ig, CD80-Ig and CD86-Ig proteins were produced in CHO cells and purified as described in A. Truneh et al., Mol Immunol, 33: 321-334 (1996) and in I. Kariv et al., J Immunol, 157: 29-38 (1996). The CTLA4-Ig protein was produced and purified in a similar manner, using the vector construct described above in part A of this section. The Igαtp fusion proteins were shown to be produced upon transfection of the Fcatplink vectors into COS-7 cells following standard procedures for transfection of COS cells (eg., Current Protocols in Molecular Immunology, edited by F. M. Ausubel et al. 1988, John Wiley & Sons, vol 1, section 9.1) and for immunoblot analysis (eg., JR Jackson et al., J. Immunology, 154:3310-3319 (1995)) with rabbit polyclonal anti-sera prepared against various derivatives of the CD80, CD86, and CTLA4 proteins or goat anti-human Fc antibody. The αtp and μtp constructs of CD80-Ig were compared in terms of their efficiency of expression and oligomerization. As determined by SDS/PAGE and immunoblot analysis, the CD80-Igμtp construct did not express as well as the αtp construct of the invention (not shown). The αtp and μtp proteins were purified from the COS cell supernatants by capture on Prosep A (Bioprocessing, Ltd., Consett County Durham, U.K.) and their state of oligomerization examined by analytical size exclusion chromatography on a 3.2×30 mm Superose 6 column run on a Smart System HPLC (Pharmacia Biotech, Piscataway N.J.). Both proteins showed a similar profile of a dominant large MW species eluting in the molecular weight range of IgM, consistent with formation of a hexameric structure, and a smaller fraction that eluted at the same size as CD80-Ig itself (not shown). However, the fraction of apparent hexamer in the αtp construct was higher (about 80%) than for the μtp construct (about 60%). Both the higher level of expression and the greater efficiency of oligomer formation indicated that the αtp construct of the invention was superior to the μtp derivative. Subsequently, the CD86-Igαtp and the CTLA4-Igαtp proteins were produced in COS cells at about the same level observed for the CD80-Igαtp protein (0.1-0.2 ug/ml). The CD80- and CD86-Igαtp proteins were then produced in a CHO cell system (A. Truneh et al., Mol Immunol, 33: 321-334 (1996)) at levels of 5-10 mg/L. This level of production is comparable to other highly expressed proteins (e.g. antibodies) produced in the same manner in this system.

These results indicate that development of standard amplified CHO cell lines with high production levels of hexamer (50 mg/L or greater) is feasible. A procedure for transfection and amplification in CHO cells is described in P. Hensley et al., J. Biol. Chem., 269:23949-23958 (1994)). Briefly, a total of 30 ug of linearized plasmid DNA (e.g. CD80Fcatplink) is electroporated into 1×10⁷ cells. The cells are initially selected in nucleoside-free medium in 96 well plates. After three to four weeks, media from growth positive wells is screened for expression—e.g., in an ELISA format using an antibody directed against the Fc region of human IgG1. The highest expressing colonies are expanded and selected in increasing concentrations of methotrexate for amplification of the transfected vectors. If a commercial vector like pBK-CMV (noted above) is used, a dhfr gene should be introduced into this plasmid or provided on a second co-transfecting plasmid to allow selection of amplification in methotrexate.

The proteins produced in CHO cells were purified by protein A affinity and size exclusion chromatography. For the CD80-Ig hexamer, thirty liters of conditioned medium containing CD80-Igαtp were chromatographed on a Protein A Sepharose Fast Flow column (Pharmacia) at 20 ml/min. The column (5.0×11.6 cm; 225 ml) were preequilibrated in 20 mM sodium phosphate, 150 mM NaCl, pH 7.5 (PBS). After loading, the column was washed with 1.8 L of PBS to baseline absorbance. CD80-Igαtp was eluted with 0.1 M sodium citrate, pH 3.0 at 10 ml/min. The eluate was neutralized immediately with 1 M Tris-HCl, pH 8.0. After filtration with a Sterivex GV filter (Millipore) using a 60 ml syringe, CD80-Igαtp was concentrated using an Amicon stirred cell and a YM100 membrane to 1.3 mg/ml. CD80-Igαtp was frozen using a dry ice ethanol bath and stored at −70° C.

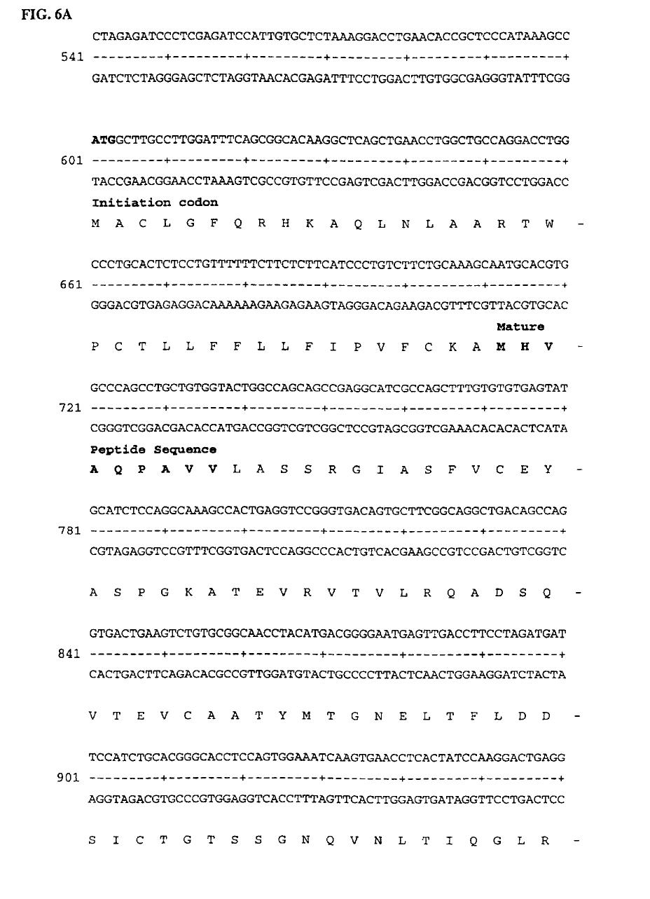

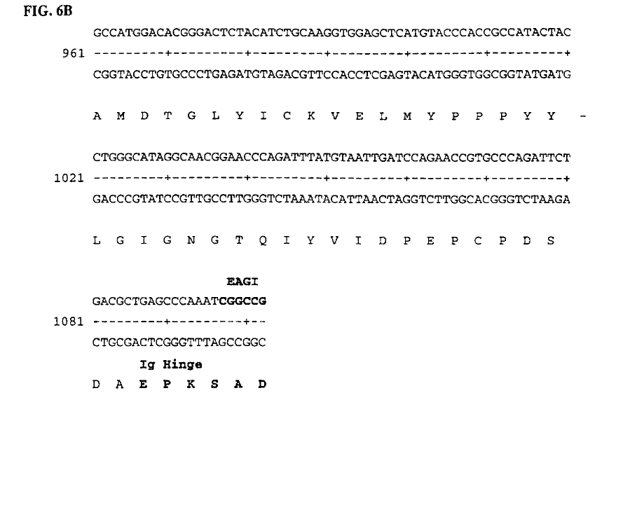

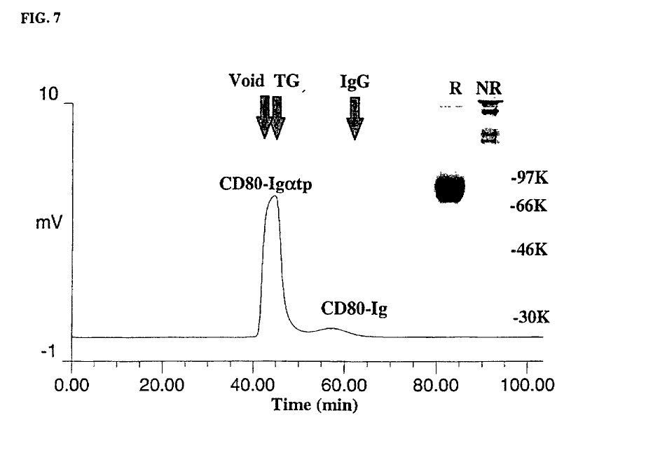

To separate hexamer from monomer, 10 ml of the concentrated CD80-Igαtp was chromatographed on a Superdex 200 column (2.6×60 cm; Pharmacia) at 2.5 ml/min. The first peak (eluted at about 45 minutes) containing the majority (about 90%) of the 280 nm absorbing material was pooled (20 ml; 0.6 mg/ml), frozen as before and stored at −70 (FIG. 7). This material eluted at approximately the position of thyroglobulin (−700,000 Da.) just behind the void volume. A minor peak at about 57 minutes corresponded to “monomer” CD80Ig. The integrity of the CD80-Igαtp in the peak fractions is shown by the single band observed in coomassie stained SDS/PAGE gel run under reducing conditions (lane R in the inset in FIG. 7). The diffuse nature of the band is characteristic of highly glycosylated proteins and is thus expected for CD80-Igαtp which contains 10 consensus N-linked glycosylation sites per polypeptide chain. Under nonreducing conditions, all of the protein migrates as high molecular weight species (lane NR in FIG. 7, insert). This dominant fraction migrated as a symmetrical peak at a MW consistent with a hexamer with a lesser amount of a species that migrated at the size observed for the monomeric CD80-Ig protein (i.e., the Ig homodimer). N-terminal amino acid sequence analysis revealed identity to the previous analysis of CD80-Ig and to that described by others [G. J. Freeman et al., 174(3):625-631 (1991)]. The CD86-Igαtp protein was purified in a similar manner. The CTLA4-Igαtp protein was expressed in COS cells, but not further characterized.

D. Protein Characterization—Molecular Size

The size exclusion chromatography noted above during purification was consistent with formation of a homogeneous hexameric species containing six CD80-Ig subunits. The size and homogeneity of the CD80-Igαtp protein produced in CHO cells was also investigated by analytical ultracentrifugation. Equilibrium sedimentation data for CD80-Igαtp in PBS, pH 7.4 is shown in FIG. 8, lower panel. The sample was sedimented at 6000 rpm for 87 hours at 25° C. in a Beckman XL-A analytical ultracentrifuge. The weight average molecular weight for a fit to all the data was 1,125,000+/−5,000 Da. The expected molecular mass of the hexamer of 864,000, assuming 2000 Da. for each N-linked glycosylation site. The data could also be fitted to a hexamer <-> (hexamer)₂ model with a K_{d} of ˜2×10⁻⁷ M. The curves in the lower panel are for the fitted distribution of hexamer and (hexamer)₂. The sum of these two curves fits the observed data well. Inclusion of terms for a monomer (131 kDa) did not improve the fit. The distribution of residuals (fitted-observed data) for the fit of the monomer dimer model to the data is shown in the upper panel of FIG. 8. The residuals are small and random, indicating a good fit. For a description of the analysis see W. Chan et al., Folding and Design, 1(2): 77-89 (1996). The lower panel inset shows g(s*) analysis of velocity sedimentation data of the protein taken in the absorption mode. Data was collected at 30,000 rpm at 22° C. The data could be fitted to two species, one of 19.4 S and one of 26.7 S which could be the hexamer and (hexamer)₂ species. For g(s*) data analysis, see W. F. Stafford, Current Opinion in Biotechnology, 8(1): 14-24 (1997).

The size and extent of covalent association of the CD80- and CD86-Igαtp proteins were examined by SDS/PAGE. Under reducing conditions all of the protein migrated in a diffuse band at about the same size as the corresponding standard Ig constructs, as shown for the CD80-Igαtp protein in the inset in FIG. 7. Under nonreducing conditions in a 4% gel, the Igαtp constructs migrated as very diffuse bands in the size range of IgM with little material co-migrating with the corresponding Ig constructs at about 150,000 Da (not shown). These results indicate that most of the individual polypeptide chains in the Igαtp proteins are covalently joined through cystine bonds, consistent with the described disulfide bond formation among the cysteine residues in the μ tailpiece segment of IgM [A. C. Davis et al., EMBO J., 8(9): 2519-2526 (1989)]. The diffuse nature of the high molecular weight bands may reflect incomplete disulfide bond formation but also is expected since a hexamer form of CD80- or CD86-Igαtp would contain 120 potential N-linked glycosylation sites.

E. Protein Characterization—Binding Properties

In several assay formats the hexameric CD80- and CD86-Igαtp proteins were distinguished from the corresponding standard Ig fusion proteins by their markedly higher binding avidity to CD28 when it was presented in a multivalent array.

1) Binding to Immobilized CD28-Ig in an ELISA Format

For this assay format, the CD80-Igαtp protein was biotinylated for simplicity of assay and for ease of detection since the CD28 protein absorbed to the plate wells was also a human Ig fusion construct. Biotinylation was carried out essentially as described in Avidin-Biotin Chemistry: A handbook, M. D. Savage et al., Pierce Chemical Company (1992). In several preparations of the protein, the molar ratio of biotin/CD80-Ig monomer was about 10:1. All steps of the assay after coating were carried out at room temperature.

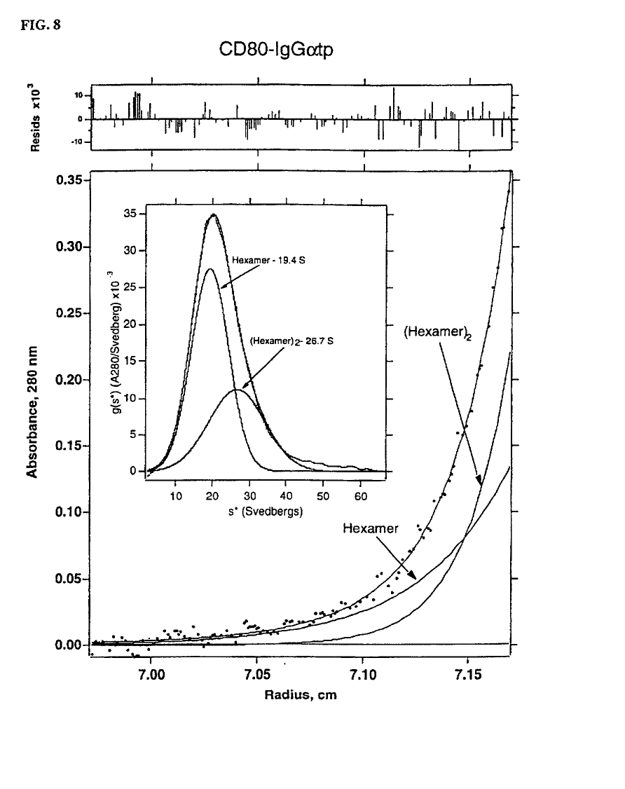

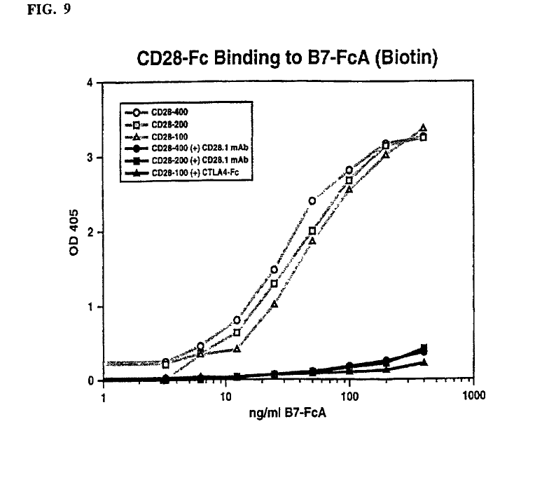

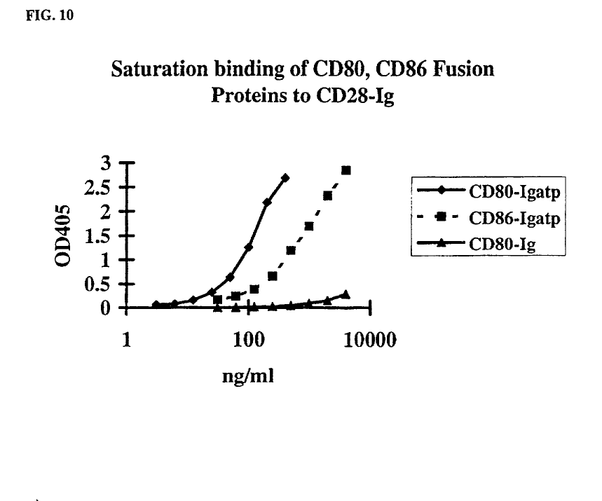

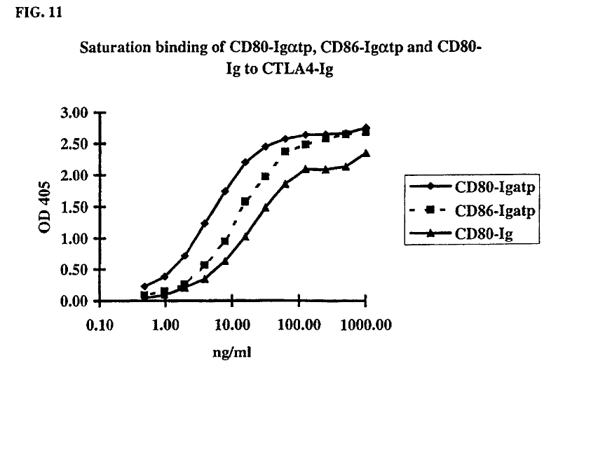

The wells of 96 well microtiter plates (Immunlon 4, Dynatech Laboratories) were coated with CD28-Ig (1, 2, or4 μg/ml) in 100 μl/well of 0.1 M sodium bicarbonate, pH 9.4 and incubated overnite @4° C. The wells were washed with PBS (phosphate buffered saline) and blocked with 0.5% gelatin in PBS for 1 hour. Following an additional PBS wash, biotinylated CD80-Igαtp was serially diluted in PBS containing 1 mg/ml BSA, 0.05% Tween directly in the wells in a final volume of 0.1 ml and incubated for 1 hour. The wells were washed with PBS and bound CD80-Igαtp protein was measured by the addition of 0.1 ml of strepavidin-HRP (streptavidin conjugated with horseradish peroxidase (Southern Biotech)) at a 1:2000 dilution for 1 hour, followed by washing and color development with 100 μl ABTS substrate (Kierkegaard and Perry Laboratories Inc., Maryland) and measurement of absorbance at 405 nm. In some cases the color reactions were arrested by addition of 100 μl of 1% SDS prior to measurement of absorbance. A plot of CD80-Igαtp binding versus concentration of added protein is shown in FIG. 9. In this figure, “CD28-Fc”, “CTLA4-Fc”, and “B7-FcA” denote CD28-Ig, CTLA4-Ig, and CD80-Igαtp, respectively. These curves (FIG. 9) indicate that concentration dependent binding of biotinylated CD80-Igαtp was inhibited by simultaneous addition of the CD28.1 MAb (a murine MAb to human CD28 that inhibits binding of CD80 to CD28; Nunes et al., Int. Immunol., 5:311-315 (1993)) or CTLA4-Ig protein (here labeled as CTLA4-Fc). Under the same conditions, biotinylated CD80-Ig itself showed little binding and only at much higher concentrations (FIG. 10). In the same format biotinylated CD86-Igαtp also showed good binding to CD28-Ig (FIG. 10). All three biotinylated proteins showed good binding to immobilized CTLA4-Ig (FIG. 11), as expected because of the higher affinity of this interaction [P. S. Linsley et. al., Immunity 1: 793-801 (1994), and see part 4 of this example below], and the rank order of binding was the same as observed with immobilized CD28-Ig.

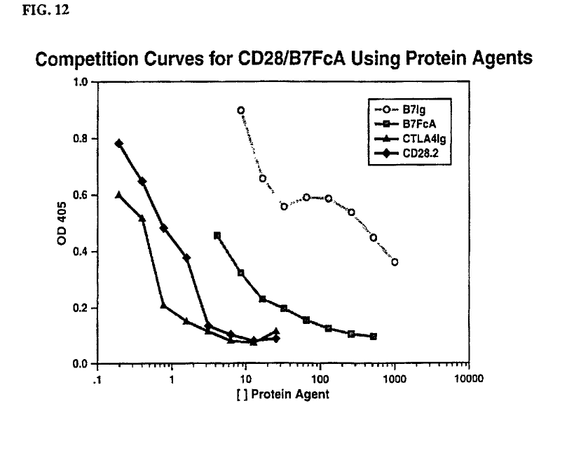

The specificity of the binding reaction was demonstrated by the expected hierarchical competition of binding with (1) CTLA4-Ig, (2) CD28.2 [Nunes et al., 1993, cited above], a murine MAb to human CD28 that inhibits binding of CD80 to CD28, (3) unlabeled CD80- and CD86-Igαtp proteins, (4) and the expected much weaker inhibition by the monomeric CD80-Ig fusion protein. One example is shown in FIG. 12. Briefly, microtiter wells were coated with 2 μg/ml CD28-Ig and biotinylated CD80-Igαtp was added at a concentration of 50 μg/ml, followed immediately by the indicated amounts of unlabeled CD80-Igαtp (B7FcA), CD80-Ig (B7Ig), CTLA4-Ig, or the MAb CD28.2. At 50 μg/ml, the biotinylated CD80-Igαtp gives about 50% saturation of OD405 (see FIG. 9). CD80-Ig was much less efficient than CD80-Igαtp in blocking binding, consistent with the expected lower affinity/avidity of the CD80-Ig protein for the immobilized CD28-Ig protein. The controls gave the expected results—the CD28.2 MAb blocked the binding site on CD28 and similarly, CTLA4-Ig blocked the binding sites on CD80-Igαtp.

Other assay formats are possible. A second example utilizes a CD28-muIg fusion protein constructed in a manner analogous to CD28-Ig except that the Ig region was derived from mouse Ig2a instead of human IgG1. More particularly, the protein was expressed using the vector CosCD28mFc2aLink, which is comparable to the CosCD28FcLink vector (described above), except that the human IgG1-Fc region was replaced with that of mouse IgG2a beginning at the Eag I site in the hinge sequence [described in I. Kariv et al., J. Immunol., 157:29-38 (1996)]. The amino acid sequence in the resulting hybrid hinge region 25 is as follows: SEQ ID NO:24—GPSKPepksagIKP—, where capital letters correspond to the end of CD28 sequence, lower case letters are residues from the human IgG₁ hinge region, underlined lower case letters are a 2 residue substitution introduced to create an Eag I site, and bold capital letters indicate the beginning of murine IgG2a hinge region.

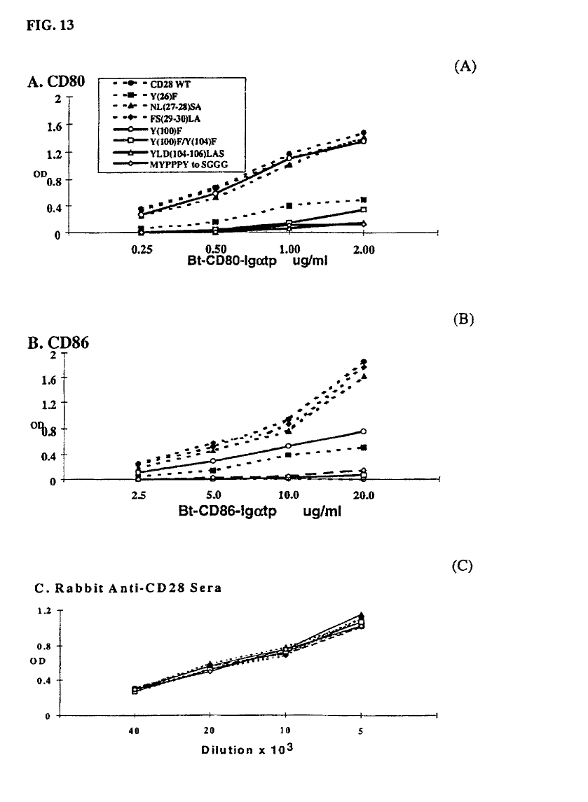

The CD28-muIg protein was indirectly immobilized in wells using goat anti-mouse Fc antibody and then CD80-Igαtp binding was carried out similarly to that described above. More specifically, CD28-muIg proteins containing wild-type or mutant CD28 sequences and, at equal concentrations, were captured on goat anti-mouse IgG antibody coated 96 well plates. The plates were washed with 1×PBS, blocked with 0.5% gelatin-PBS for 1 hour, and then incubated with either biotinylated CD80- or CD86-Igαtp for 45 min. The plates were washed and Igαtp fusion protein was quantitatedas described above. . This assay was used to examine the effects of mutations in CD28 on binding to CD80 and CD86, as illustrated in FIGS. 13A and 13B (I. Kariv et al., J. Immunol., 157:29-38 (1996)). Each of the mutant CD28-muIg2a proteins was captured on the goat anti-mouse IgG coated wells and the binding of biotinylated CD80-Igαtp (FIG. 13A) or CD86-Igαtp (FIG. 13B) was measured. Equivalent capture of each of the CD28-muIg2a proteins was verified by the comparable binding of polyclonal rabbit CD28 antisera to each of the proteins (FIG. 13C).

2) Binding to Immobilized CD28-Ig in a Biosensor Assay Format

The binding of CD80-Igαtp or CD80-Ig to immobilized CD28 were compared by surface plasmon resonance analysis using a BIAcore instrument, following procedures similar to that described for other proteins [K. Johanson et. al., J. Biol. Chem, 270: 9459-9471 (1995), and references therein].

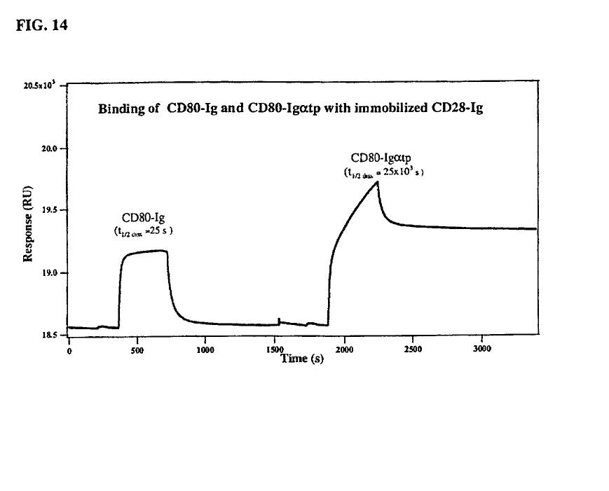

For comparison of CD80-Ig and CD80-Igαtp, approximately 4000 RU of CD28-Ig were immobilized onto a BIAcore CM5 sensor surface (BIAcore, Piscataway, N.J.) by covalent attachment to the surface through its amines. Covalent attachment was achieved by firstly activating the surface with a 1:1 mixture of 0.1 M solution of N-hydroxysuccinamide and 0.1 M 1-ethyl-3-(3-dimethylaminopropyl)carbodiimide. A solution of CD28-Ig 50 ug/ml in 0.01 M sodium acetate pH 4.7 was then passed over the surface. Unreacted N-hydroxysuccinamide esters were then deactivated with 1 M ethanolamine pH 8.5. The surface was equilibrated with running buffer composed of 20 mM HEPES 150 mM NaCl, pH 7.2, 3 mM EDTA and 0.005% Tween 20. The CD80-Ig and CD80-Igαtp (20 ug/ml) diluted in running buffer were injected over the surface (60 ul) with a flowrate of 10 ul/min. The results show that CD80-Ig dissociates very rapidly from the CD28-Ig coated surface, whereas the rate of dissociation for CD80-Igαtp is about three orders of magnitude slower (FIG. 14).

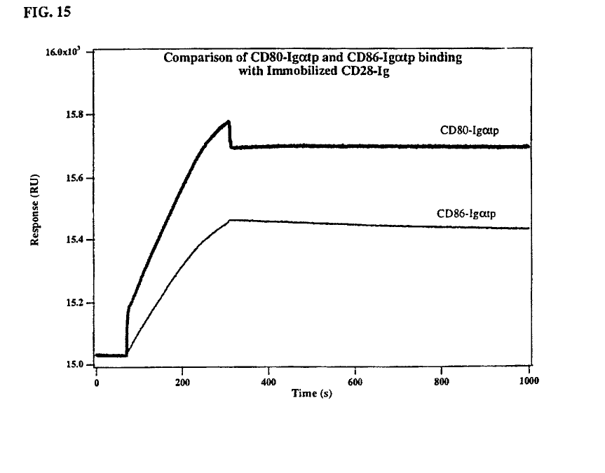

For comparison of CD80-Igαtp and CD86-Igαtp binding with immobilized CD28-Ig, solutions of CD80-Igαtp and CD86-Igαtp (5 ug/ml) were prepared in running buffer described above. Sample solutions were injected (60 ul) at 10 ul/min. Between samples, the surface was regenerated with a 30 ul injection of Gentle elution buffer (Pierce Chemicals, Rockville, Ill.). The results show that like CD80-Igαtp above, CD86-Igαtp dissociates slowly from the CD28-Ig surface (FIG. 15). The off-rate for CD86-Igαtp appears higher than that for the CD80 construct, consistent with the weaker binding of this protein in the ELISA (part 1, above) and cell binding (part 3, below) formats and the lower intrinsic affinity measured by calorimetry (part 4, below).

3) Binding to Cells Expressing Cell-surface CD28

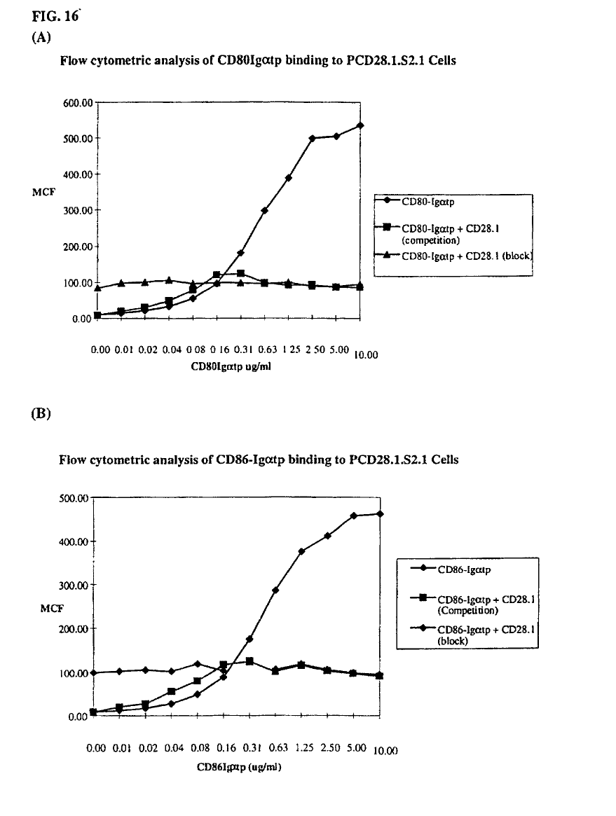

By flow cytometry, both CD80- and CD86-Igαtp show specific binding to CD28 positive cells (FIGS. 16A and 16B), whereas no binding is observed with CD80- or CD86-Ig themselves (not shown). Non-adherent CD28 expressing cells (PCD28.1.s2.1) were used for this assay format. PCD28.1.S2.1 cells were created by transfection of PE30.2 cells [D. Emilie et al., Eur. J. Immunol., 19:1619-1624 (1989)] with a vector for expression of human CD28 [D. Couez et al., Molecular Immunology, 31:47-57 (1994)]. All incubations were carried out on ice. Unlabelled CD80- and CD86-Igαtp or CD80 and CD86-Ig were incubated with the cells in binding buffer consisting of PBS w/o Ca+2/Mg+2, 0.2% bovine serum albumin and 0.1 % sodium azide. After washing twice in binding buffer, bound fusion proteins were detected with a 1:2000 dilution of a goat anti-human polyclonal antibody labeled with FITC (Flourescein isothiocyanate, Southern Biotech). Following two additional washes in binding buffer, the cells were resuspended in binding buffer and analyzed on a FACSort analyzer (Becton-Dickinson) using a 488 nm laser. Low non-specific binding was shown by incubating the cells with a 200 fold excess of a CD28 monoclonal antibody 28.1 prior to (“block” curves, Figure C), or at the same time as (“competition” curves, FIGS. 16A and 16B), or by addition of CTLA4-Ig with the CD80 and CD86 fusion proteins (not shown).

4) Binding to CD28-Ig and CTLA4-Ig in Solution

The solution binding properties of the hexameric and standard Ig fusion proteins of CD80 and CD86 were compared using isothermal titration calorimetry, essentially as described previously for other proteins [K. Johanson et. al., J. Biol. Chem, 270: 9459-9471 (1995), and references therein]. The binding to CTLA4-Ig is summarized in the following table:

TABLE I
Direct comparison of CTLA4-Ig binding properties
of CD80 and CD86 Ig fusion proteins as measured
by titration calorimetry at 2 temperatures

| | Experi- | | | | |
|---|---|---|---|---|---|
| | mental | K_{d}, nM | Molar Ratio | | K_{d} |
| | Temper- | at Temp- | (CTLA4-Ig | ΔH, | nM at |
| Construct | ature | erature | per construct) | kcal/mol | 37° C. |
| CD80-Ig | 37° C. | 5.4 | 0.72 | −32 ± 2 | 5 |
| CD80- | 37° C. | 5.9 | 4.02 | −36 ± 2 | 6 |
| Igαtp | | | | | |
| CD86-Ig | 37° C. | 38 | 0.80 | −33 ± 3 | 40 |
| CD86- | 37° C. | 20 | 2.72 | −37 ± 4 | 20 |
| Igαtp | | | | | |
| CD80-Ig | 44° C. | 4.9 | 0.60 | −36 ± 4 | 2 |
| CD80- | 44° C. | 8.1 | 3.79 | −38 ± 3 | 2 |
| Igαtp | | | | | |
| CD86-Ig | 44° C. | 71 | 0.79 | −34 ± 4 | 22 |
| CD86- | 44° C. | 38 | 2.83 | −41 ± 4 | 9 |
| Igαtp | | | | | |

The error in Kd's is about a factor of 2 and the error in molar binding ratio's is 10-20%. Kd values at 37° C. were either measured directly at 37° C. or were corrected for temperature differences using the van't Hoff equation, as described in M. L. Doyle et. al., J. Mol. Recognition, 2: 65-74 (1996). Concentrations were defined by absorbance at 280 nm using the following: A) molecular masses of 90,059 (CTLA4-Ig), 127,000 (CD80-Ig), 810,000 (CD80-Igαtp), 140,000 (CD86-Ig), and 890,000 (CD86-Igαtp) and B) calculated extinction coefficients of 1.22 (CTLA4-Ig), 1.10 (CD80-Ig and CD80-Igαtp), and 1.03 (CD86-Ig and CD86-Igαtp). The molecular weights for CTLA4-Ig, CD80-Ig, and CD86-Ig were determined by mass spectral analysis. The molecular masses of CD80-Igαtp and CD86-Igαtp were estimated as 6× the mass of the respective Ig proteins plus 40,000 Da. contributed by the twelve tailpiece segments.

Direct comparison of the CTLA4-Ig binding to CD80-Ig, CD80-Igαtp, CD86-Ig, and CD86-Igαtp constructs in solution phase by isothermal titration calorimetry demonstrates several features. First, the affinities of the Ig versus Ig-αtp constructs are equivalent in solution. This suggests that, as expected, solution binding affinities of the αtp constructs do not benefit from avidity effects like they do in ELISA and cell binding assays. Second, the enthalpy changes which accompany the molecular interactions of the Ig and Igαtp constructs are also the same and support the view that the molecular details of the interactions are the same. Third, the titration equivalence points for CTLA4-Ig binding to the CD80 and CD86 Ig versus Igαtp constructs indicate that all these reagents were ≧50% active during the calorimetry assay. With regard to this latter point, comparison of the Igαtp and Ig constructs shows a ratio of about 6 for CD80, indicating about equivalent binding activity for the CD80 domains in both constructs. The lower ratio for the corresponding CD80-Igαtp protein indicates some loss of activity in this preparation.

Interactions of CD28-Ig with either CD80- or CD86-Ig were not detected in solution by calorimetry, suggesting an affinity of interaction weaker than 1 uM. This lower affinity for CD28 than for CTLA4 is in agreement with other reports [P. S. Linsley et. al., Immunity 1: 793-801 (1994)]. CD28-Ig also did not show detectable binding to CD80- or CD86-Igαtp, which is consistent with the solution affinities of the αtp constructs not benefiting from avidity effects.

### EXAMPLE 2

### Demonstration of Agonist Activity for the CD80- and CD86-Igαtp Protein

A. CTLL-2 Bioassay for Detection of IL-2 Levels

The CD80- and CD86-Igαtp proteins were compared to the corresponding CD80 and CD86-Ig proteins to determine their ability to stimulate cells expressing human CD28 using two murine T-cell hybridoma cell lines expressing human CD28, PCD28.1.s2.1 and DCL27CD28wt.s2. The PCD28.1.s2.1 cell line was described in Example 1, part 3. The DCL27CD28wt.s2 cell line was created by transfection of the DC27 cell line [F. Pages et al., Nature, 369:327-329 (1994); F. Pages et al., J. Biol. Chem., 271(16):9403-9409 (1996)] with the same CD28 expression vector used for the PCD28.1.s2.1 cells [D. Couez et al., Molecular Immunology, 31:47-57 (1994)]. These cell lines were examined for their ability to produce IL-2 in response to activation with CD80- and CD86-Ig in comparison with the corresponding Igαtp fusion proteins. 96-well plates were coated with or without a CD3 antibody together with the CD80 and CD86 fusion proteins. This was accomplished by first incubating the plates with a previously determined suboptimal concentration of hamster anti-human CD3 antibody (MAb 145-2C11, Boerhinger-Mannheim Biochemicals) for two hours at room temperature (RT) or with just buffer alone, washing the plates with PBS, adding goat anti-human Ig heavy chain (GAH-IgHc, Sigma Chemical Co.) for an additional two hours at RT, washing again and coating with different concentrations of the CD80 or CD86 fusion protein for 16-18 hours at 4° C., washing again, and finally blocking for 30 min. with 0.2% BSA-PBS. T cells (1×10⁵/well) were added in 150 μl medium into duplicate wells. For comparison, the soluble fusion proteins and the 248.23.2 CD28 MAb (IgM) [A. Morretta, University of Genova, Italy] were added to non-coated wells. T cells were incubated in the wells for 24 hours at 37° C., and supernatants were collected and evaluated for IL-2 levels in a standard CTLL-2 bioassay [S. M. Gillis et al., J. Immunol., 120:2027 (1978)]. Briefly, 1×10⁴ IL-2 dependent CTLL-2 cells (ATCC)/well in 75 μl medium were added to an equal volume of test supernatant and incubated for 24 hours at 37° C. The cells were pulsed with 10 μl of 5 mg/ml MTT (Sigma Chemical Co.) for 4 hours, and lysed with 100 ml 10% SDS/0.01N HCl solution for 14-16 hours. OD₅₇₀ readings were converted into ng/ml of IL-2 based on a standard curve generated by treating cells with known concentrations of IL-2.

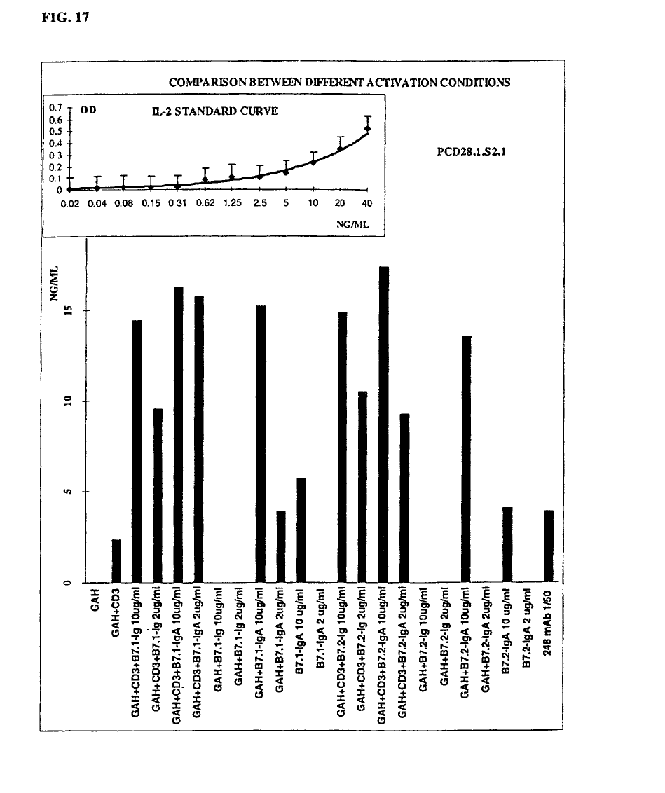

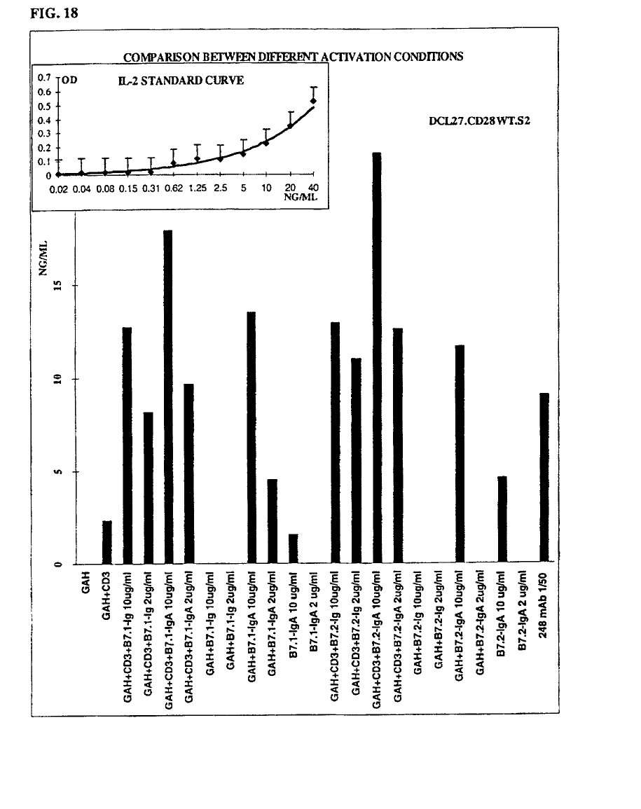

In all assays, the CD80- and CD86-Igαtp proteins were more efficient stimulators of the CD28 T-cells than the corresponding monomeric Ig constructs (FIGS. 17 and 18). The soluble hexameric proteins induced IL-2 production in the absence of CD3 crosslinking (GAH), whereas under the same conditions, no activity was observed with CD80- or CD86-Ig themselves. A similar level of IL-2 induction was observed with the oligomeric CD28 IgM antibody 248.23.2. Cross-linking of the hexameric CD80 and CD86 proteins with GAH antibody increased the IL-2 response relative to the absence of cross-linker, but still did not give a response for the monomeric Ig constructs. In the presence of CD3 antibody, the differences between the hexameric and monomeric Ig fusion proteins were minimal, being about 2-fold or less.

B. Fluorescein di-b-D-galactosidase (FDG) Assay for Detection of IL-2 Promoter Activity

A second assay for agonist activity measured induction of IL-2 promoter activity, rather than production of IL-2 protein. The PCD28.1.S2.1 cells described above also contain lacZ fused to the IL-2 promoter. Thus, the PCD28.1.S2.1 cell line provides a convenient system for measuring IL-2 promoter activity upon CD28-mediated T cell simulation. T cells were activated as described above for the CTLL-2 assay, spun down, resuspended in 50 μl of media+50 μl of PBS, lysed with 10 μl of 20% Triton X-100, and supplemented with 25 μl of 10 mM FDG (Molecular Probes), a fluorogenic substrate for b-galactosidase. Hydrolysis of FDG first yields fluorescein monogalactoside (FMG) and then the highly fluorescent product fluorescein. Cell lysates were incubated with FDG for 60 min., and the levels of fluorescence were measured by Fluoroscan (MTX Lab Systems, Inc).

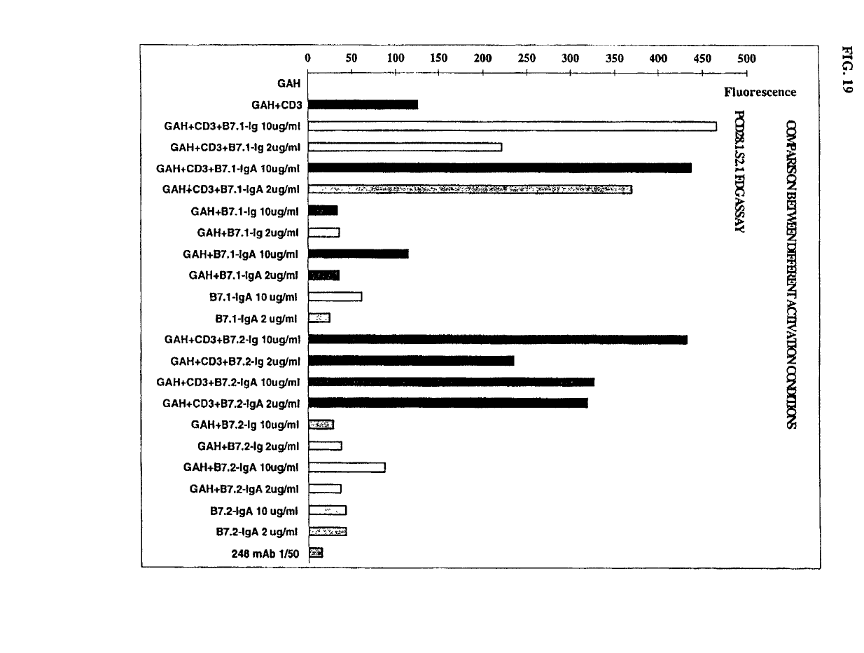

The results of these assays (FIG. 19) were similar to those described above for IL-2 production. The primary difference was that low levels of IL-2 promoter activity were observed for the monomeric Ig proteins.

C. Stimulation of CD28 Cells By CD80- and CD86-Igαtp Proteins in Solution

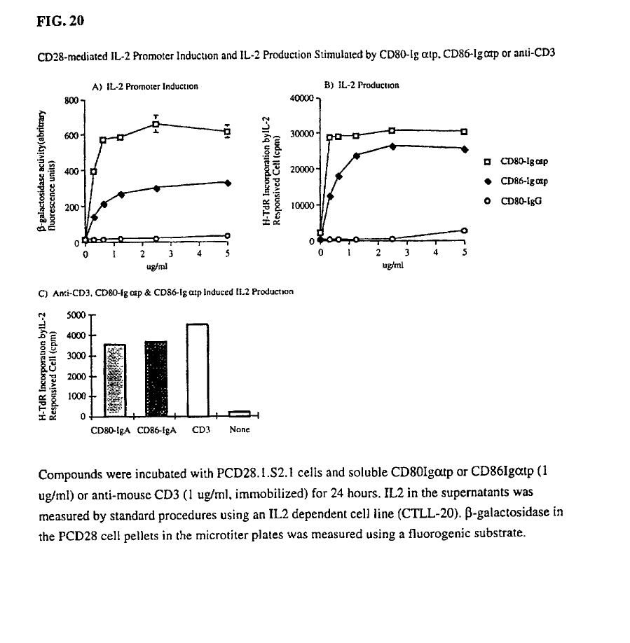

In the above examples (parts A and B), the Ig and Igαtp proteins showed the greatest activity when captured on the surface of the microtiter well.. However, the CD80- and CD86-Igαtp proteins were also able to stimulate CD28 cells when added directly to the cells in solution, whereas no response was observed with the corresponding standard Ig fusion proteins. CD80-and CD86-Igαtp showed a dose dependent stimulation of IL-2 promoter activity (FIG. 20A) and IL-2 production (FIG. 20B) when added to PC28.1.s2.1 cells. In contrast, no stimulation was observed with CD80-Ig (FIGS. 20A and 20B) or CD86-Ig (not shown). IL-2 promoter activity and IL-2 levels were measured similarly to that described in parts A and B above, except that proliferation of the reader CTLL-2 cells was measured by ³H-thymidine incorporation. The level of response at near saturation levels of CD80- and CD86-Igαtp proteins (1 ug/ml) was comparable to that observed for stimulation through cross-linking of CD3 with immobilized CD3 antibody (FIG. 20C). The specificity of the response to CD80- and CD86-Igαtp was confirmed by complete blockade with the addition of CTLA4-Ig (not shown).

In summary, the results from these assays show that the CD80- and CD86-Igαtp proteins have agonist activity under conditions where little or no activity was observed for the corresponding monomeric Ig proteins.

### EXAMPLE 3

### Compound Screen Assay for Identifying Small Molecule Antagonists of the Interaction Between CD28 and CD80

An ELISA format was used to identify small molecule antagonists of CD80 and CD86 binding to CD28 by screening a large bank of chemical compounds and natural products. The assay was carried out as in the format described in Example l, part E.1, except that immediately following addition of the biotinylated CD80-Igαtp (222 ng/ml in a volume of 90 μl), dilutions of test compound were added (10 μl). The compounds were dissolved at 100× assay concentration in dimethyl sulfoxide (DMSO) and subsequently diluted in 50%DMSO/50% H₂O to a 10× working stock. The assay was not sensitive (<10% alteration of signal) to DMSO at concentrations of 5% or less.

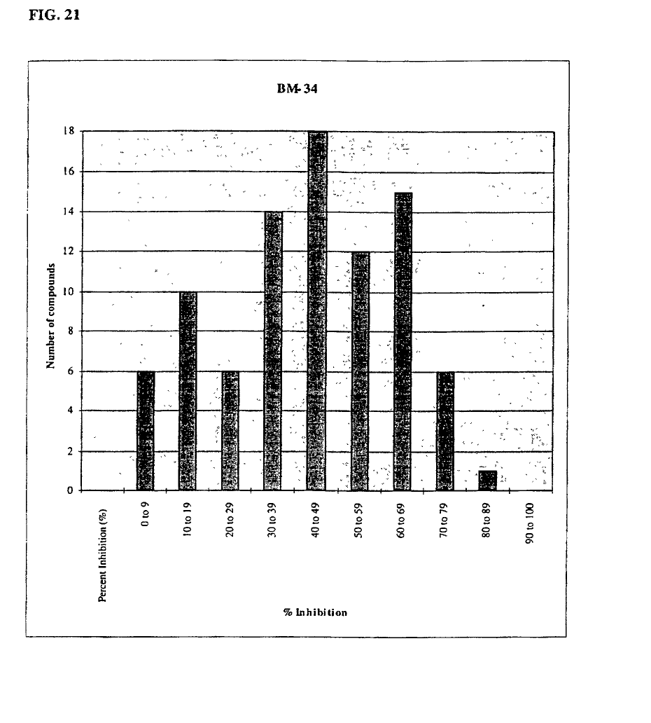

Results from one test assay are summarized in FIG. 21 and Table II. The BM-34 test set consists of 968 compounds in two formats—as individual compounds and as 88 multimixes with 11 individual compounds in each multimix sample. Both BM-34 formats were assayed (at a concentration of 200 μg/ml for each multimix sample and 20 μg/ml for individual compounds) for inhibition of biotinylated CD80-Igαtp binding to immobilized CD28-Ig in 96 well plates. Results for setting a 70% or 85% cutoff for inhibition are shown in Table II. In FIG. 21, the percent inhibition range is plotted against the number of compounds showing the indicated range of inhibition. The low percentage of compounds showing activity in the 80-90% range of inhibition makes this a suitable threshold for rapid screening.

TABLE II
CD80-Igαtp Screen Assay Results
BM-34 Test Compound Set

| Result | 70% Cutoff | 85% Cutoff |
|---|---|---|
| Hits on Multimix plates | 7 | 1 |
| + Multimix Samples with | 5/7 | 1/1 |
| + Compound | | |
| − Multimix Samples with | 8 | 0 |
| + Compound | | |

As illustrated in this table, eight of the mixes gave 70% or greater inhibition. Deconvolution by assay of the individual compounds from these 8 mixes at 20 μg/ml confirmed that there was a compound with comparable activity. The two other mixes that failed to confirm had one or more compounds with activity very close to the 70% inhibition observed in the original multimix assay. Selecting a higher cutoff of 85% gave only one multimix hit and that was confirmed in the assay of individual compounds. Further evidence of reproducibility and selectivity was that only eight compounds from mixes below the 70% cutoff showed >70% inhibition when assayed individually. Selectivity was further increased by reducing the concentration of the multimix samples to 100 μg/ml and the individual compounds to 10 μg/ml. This corresponds to about a 30 μM concentration for the compounds since their average MW is 300-400 daltons. At 100 μg/ml, multimix samples showed a desired shift to lower average inhibition (90% of the mixtures gave 60% or less inhibition) while retaining an acceptable hit rate at a high level of inhibition (4% of the mixtures giving 70% or greater inhibition). Through the use of this assay, small molecule inhibitors of the interaction of CD80 with CD28 can be identified.

### EXAMPLE 4

### αtp-mediated Oligomerization of a Mouse/Human IgG1 Chimeric Antibody

To examine the generalization of αtp-mediated hexamer formation of the Fc region of human IgG, the αtp segment was introduced into a chimeric antibody containing heavy and light chain variable regions from the mouse monoclonal antibody 1C8 and the human kappa and IgG1 constant regions. 1C8 is directed against the human EPO (erythropoeitin) receptor. The αtp sequence was introduced onto the heavy chain of the antibody by replacing the Eco RI/Sac II fragment of CD80Fcαtplink with the Eco RI/Sac II fragment of EpoR(CH)IgG1-PCN, a vector containing the heavy chain of the chimeric 1C8 antibody, to give the vector EpoR(CH)Fcαtplink. In both vectors, Eco RI cleaves between the CMV promoter and the start of the N-terminal signal sequences and Sac II cleaves at a conserved site in constant region 2 of the human heavy chain.

Test samples of the hexameric mAb were produced in COS-7 cells upon co-transfection of EpoR(CH)Fcαtplink and a vector for expression of the light chimeric light chain, following procedures described above in Example 1, part C. Initially, 5 T150 flasks were co-transfected with the two vectors and 300 ml of conditioned media were collected. The hexameric antibody was purified by affinity chromatography on Protein A. Purity was about 90% as determined by coomassie staining of the sample as analyzed by reducing SDS/PAGE. Under nonreducing conditions on SDS/PAGE, the antibody migrated in the size range of IgM (not shown).

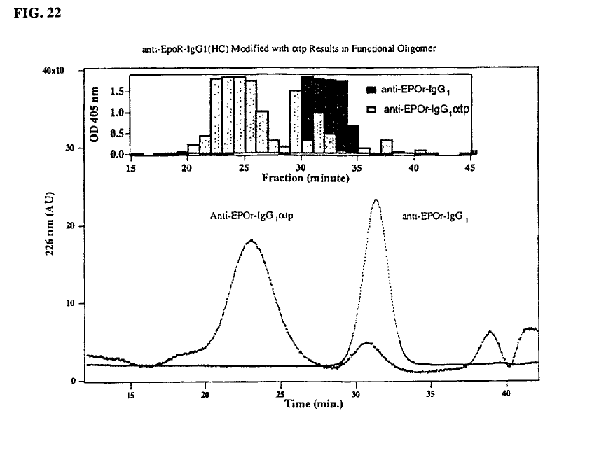

The sample was further characterized by analytical size exclusion chromatography on a 3.2×30 mm Superose 6 column run on a Smart System HPLC (Pharmacia Biotech, Piscataway N.J.). The major peak (FIG. 22) corresponds to binding activity, as monitored in an ELISA using a recombinant human EPO receptor Ig fusion protein (EPOr-Ig), and eluted at a size consistent with hexamer formation (anti-EPOr-IgG₁ αtp). The parental chimeric antibody (anti-EPOr-IgG₁) elutes substantially later from the column and is represented in the figure by the dashed lines.

These results indicate that addition of the αtp segment to the human IgG1 constant region leads to formation of hexameric antibody.

Numerous modifications and variations of the present invention are included in the above-identified specification and are expected to be obvious to one of skill in the art. Such modifications and alterations to the compositions and processes of the present invention are believed to be encompassed in the scope of the claims appended hereto.

What is claimed is:

1. A hexameric fusion protein comprising:
(a) a dimeric binding protein and
(b) a tailpiece αtp) characterized by having the activity of the tailpiece from the C-terminus of the heavy chain of an IgA antibody.

2. The fusion protein according to claim 1, wherein the dimeric binding protein is selected from the group consisting of:
(a) a protein fragment comprising the extracellular domain of a selected monomeric binding protein or a functional fragment thereof fused to an Ig-Fc fragment selected from the group consisting of an Fc fragment from an IgG antibody, an Fc fragment from an IgD antibody, an Fc fragment from an IgE antibody, and an Fc fragment from an IgM antibody excluding the μtp; and
(b) a naturally dimeric binding protein or a fragment thereof having the binding ability of said dimeric protein.

3. The fusion protein according to claim 2 further comprising a leader suitable for expression and processing of the fusion protein.

4. The fusion protein according to claim 2 wherein the protein fragment consists of the native leader and extracellular domains selected from the group consisting of CD80, CTLA-4 and CD86.

5. The fusion protein according to claim 2 wherein the dimeric binding protein is a Ig-Fab fragment and the heavy chain is joined to the Ig-Fc fragment.

6. The fusion protein according to claim 2 wherein the Ig-Fc fragment is from an IgG antibody selected from the group of human isotypes consisting of IgG₁, IgG₂, IgG₃, IgG₄, and IgG binding mutants.

7. The fusion protein according to claim 2 wherein the Ig-Fc fragment is from a human IgG₁ antibody.

8. The fusion protein according to claim 1 wherein the αtp is the tailpiece of an antibody selected from the group consisting of human IgA1, human IgA2, rabbit IgA, mouse IgA, and gorilla IgG.

9. The fusion protein according to claim 8 wherein the αtp has the sequence
SEQ ID NO: 10 PTHVNVSVVMAEVDGTCY.

10. The fusion protein according to claim 8 wherein the αtp has been modified to remove the N-linked glycosylation site.

11. The fusion protein according to claim 1 further comprising a linker of between 1 to about 20 amino acids in length, said linker located between the binding protein and the αtp.

12. The fusion protein according to claim 1 which is a homo-hexamer.

13. The fusion protein according to claim 1 which is a hetero-hexamer.

14. A polynucleotide sequence encoding a hexameric fusion protein comprising:
(a) a dimeric binding protein and
(b) a tailpiece (αtp) characterized by having the biological activity of the tailpiece from the C-terminus of the heavy chain of an IgA antibody.

15. The polynucleotide sequence according to claim 14, wherein the dimeric binding protein is selected from the group consisting of:
(a) a protein fragment comprising the extracellular domain of a selected monomeric binding protein fused to an Ig-Fc fragment selected from the group consisting of an Fc fragment from an IgG antibody, an Fc fragment from an IgD antibody, an Fe fragment from an IgE antibody, and an Fc fragment from an IgM antibody excluding the μtp; and
(b) a naturally dimeric binding protein or a fragment thereof having the binding ability of said protein.

16. The polynucleotide sequence according to claim 15 further comprising a leader suitable for expression and processing of the fusion protein.

17. The polynucleotide sequence according to claim 15 wherein the protein fragment consists of the native leader and extracellular domains selected from the group consisting of CD80, CTLA-4 and CD86.

18. The polynucleotide sequence according to claim 15 wherein the dimeric protein is a Ig-Fab fragment and the heavy chain is joined to the Ig-Fc fragment.

19. The polynucleotide sequence according to claim 15 wherein the Ig-Fc fragment is from an IgG antibody selected from the group of human isotypes consisting of IgG₁, IgG₂, IgG₃, IgG₄, and IgG binding mutants.

20. The polynucleotide sequence according to claim 15 wherein the Ig-Fc fragment is from a human IgG₁ antibody.

21. The polynucleotide sequence according to claim 14 wherein the αtp is the tailpiece of an antibody selected from the group consisting of human IgA1, human IgA2, rabbit IgA, mouse IgA, and gorilla IgG.

22. The polynucleotide sequence according to claim 21 wherein the αtp has the sequence
SEQ ID NO: 10 PTHVNVSVVMAEVDGTCY.

23. The polynucleotide sequence according to claim 21 wherein the αtp has been modified to remove the N-linked glycosylation site.

24. The polynucleotide sequence according to claim 14, wherein the fusion protein further comprises a linker of between 1 to about 20 amino acids in length, said linker located between the binding protein and the αtp.

25. A vector comprising a polynucleotide sequence encoding:
(a) a polynucleotide sequence according to claim 14; and
(b) sequences controlling expression of the fusion protein in a selected host cell.

26. A recombinant host cell comprising the vector of claim 25.

27. A pharmaceutical composition comprising an hexameric fusion protein according to claim 1 in a pharmaceutically acceptable carrier.

28. A pharmaceutical composition comprising a polynucleotide sequence according to claim 14 in a pharmaceutically acceptable carrier.

29. A diagnostic reagent comprising a delectable label and an hexameric fusion protein according to claim 1.

30. A diagnostic reagent comprising a detectable label and a polynucleotide sequence according to claim 14.

31. A method for producing a hexameric fusion protein comprising the steps of:
(a) providing a dimeric binding protein; and
(b) attaching to each monomer of said binding protein a tailpiece (αtp) characterized by having the biological activity of the tailpiece from the C-terminus of the heavy chain of an IgA antibody.

32. A method of purifying a hexameric fusion protein comprising:
(a) providing a selected host cell according to claim 26;
(b) recovering the stable hexameric fusion protein; and
(c) purifying the recovered fusion protein.

33. The method according to claim 32, wherein said fusion protein comprises IgG or a fragment thereof, and said purification step comprises the step of applying said fusion protein to a Protein A or Protein G column.

34. A method for screening for a ligand which binds to a hexameric fusion protein according to claim 1, comprising the steps of:
(a) providing the hexameric fusion protein;
(b) permitting a test sample to come into contact with the hexameric fusion protein; and
(c) detecting binding between the fusion protein and any ligand in the test sample.

35. The method according to claim 34 wherein the fusion protein is immobilized to a surface.

36. The method according to claim 34 wherein the fusion protein is in solution.

37. The method according to claim 34, wherein the fusion protein is selected from the group consisting of CD80-Igαtp and CD86-Igαtp.

38. A method for screening for a compound that inhibits the interaction between a selected binding protein and a ligand, said method comprising the step of (a) providing a known ligand for said binding protein;
(b) providing a hexameric fusion protein according to claim 1;
(c) contacting the known ligand with a test solution;
(d) contacting the known ligand with the hexameric fusion protein;
(e) detecting inhibition of binding of the hexameric fusion protein; and
(f) optionally isolating the compound which binds to the hexameric protein.

39. The method according to claim 38, wherein the ligand is selected from the group consisting of CD28 and CTLA4 and the hexameric fusion protein is selected from the group consisting of CD80-Igαtp and CD86-Igαtp.

40. A method for stimulating CD28 positive cells comprising the step of administering to CD28 positive cells a hexameric fusion protein selected from the group consisting of CD80-Igαtp and CD86-Igαtp.

41. A method for suppressing CTLA-4 positive cells comprising the step of administering to CTLA-4 positive cells a hexameric fusion protein selected from the group consisting of CD80-Igαtp and CD86-Igαtp.

42. A method for antagonizing cell surface CD80- and CD86-mediated stimulation of CD28 positive cells by administering to said cells a hexameric fusion protein CTLA4-Igαtp.

43. A method for immunizing an animal comprising the method of administering to the animal an effective amount of a pharmaceutical compositions according to claim 27.

44. A method for immunizing an animal comprising the method of administering to the animal an effective amount of a pharmaceutical compositions according to claim 28.


SEQUENCE LISTING (27 sequences)

SEQ ID NO: 1 - DNA, 7167 bp, HOMO SAPIENS
gacgtcgacggatcgggagatcggggatcgatccgtcgacgtacgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgggtacgtgaaccgtcagatcgcctggagacgccatcgaattcggttacctgcagatatcaagctaattcggtaccgggcccccctcgagcctgaagccatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgatcaggagcccaaatcggccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctaagcttgtctgcgggtaaacccacccatgtcaatgtgtctgttgtcatggcggaggtggacggcacctgctactgatagtctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgaggggggatctcccgatccccagctttgcttctcaatttcttatttgcataatgagaaaaaaaggaaaattaattttaacaccaattcagtagttgattgagcaaatgcgttgccaaaaaggatgctttagagacagtgttctctgcacagataaggacaaacattattcagagggagtacccagagctgagactcctaagccagtgagtggcacagcattctagggagaaatatgcttgtcatcaccgaagcctgattccgtagagccacaccttggtaagggccaatctgctcacacaggatagagagggcaggagccagggcagagcatataaggtgaggtaggatcagttgctcctcacatttgcttctgacatagttgtgttgggagcttggatagcttggacagctcagggctgcgatttcgcgccaaacttgacggcaatcctagcgtgaaggctggtaggattttatccccgctgccatcatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccaccttagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgctagcttgaacttgtttattgcagcttataatggttacaaataaagcaatagcatcacaaatttcacaaataaagcatttttttcactgcattctagttgtggtttgtccaaactcatcaatgtatcttatcatgtctggatcaacgatagcttatctgtgggcgatgccaagcacctggatgctgttggtttcctgctactgatttagaagccatttgccccctgagtggggcttgggagcactaactttctctttcaaaggaagcaatgcagaaagaaaagcatacaaagtataagctgccatgtaataatggaagaagataaggttgtatgaattagatttacatacttctgaattgaaactaaacacctttaaattcttaaatatataacacatttcatatgaaagtattttacataagtaactcagatacatagaaaacaaagctaatgataggtgtccctaaaagttcatttattaattctacaaatgatgagctggccatcaaaattccagctcaattcttcaacgaattagaaagagcaatctgcaaactcatctggaataacaaaaaacctaggatagcaaaaactcttctcaaggataaaagaacctctggtggaatcaccatgcctgacctaaagctgtactacagagcaattgtgataaaaactgcatggtactgatatagaaacggacaagtagaccaatggaatagaacccacacacctatggtcacttgatcttcaacaagagagctaaaaccatccactggaaaaaagacagcattttcaacaaatggtgctggcacaactggtggttatcatggagaagaatgtgaattgatccattccaatctccttgtactaaggtcaaatctaagtggatcaaggaactccacataaaaccagagacactgaaacttatagaggagaaagtggggaaaagcctcgaagatatgggcacaggggaaaaattcctgaatagaacagcaatggcttgtgctgtaagatcgagaattgacaaatgggacctcatgaaactccaaagctatcggatcaattcctccaaaaaagcctcctcactacttctggaatagctcagaggccgaggcggcctcggcctctgcataaataaaaaaaattagtcagccatgcatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattaattcccgatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacct

SEQ ID NO: 2 - DNA, 1560 bp, HOMO SAPIENS
  CDS (63)...(1538)
gaattcggttacctgcagatatcaagctaattcggtaccgggcccccctcgagcctgaagccatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgatcaggagcccaaatcggccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctaagcttgtctgcgggtaaacccacccatgtcaatgtgtctgttgtcatggcggaggtggacggcacctgctactgatagtctagagctcgctgat

SEQ ID NO: 3 - protein, 492 aa, HOMO SAPIENS
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDQEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSAGKPTHVNVSVVMAEVDGTCY

SEQ ID NO: 4 - DNA, 831 bp, HOMO SAPIENS
  CDS (52)...(831)
tcggttacctgcagatatcaagctaattcggtaccagcagaagcagccaaaatggatccccagtgcactatgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtattatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacgagcccaaatcggccgacaaaactcacacatgcccaccgtgccca

SEQ ID NO: 5 - protein, 260 aa, HOMO SAPIENS
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHEPKSADKTHTCPPCP

SEQ ID NO: 6 - DNA, 1104 bp, HOMO SAPIENS
  CDS (601)...(1104)
gacgtcgacggatcgggagatcggggatcgatccgtcgacgtacgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcgactcactataggagttcccaagcttctagagatccctcgagatccattgtgctctaaaggacctgaacaccgctcccataaagccatggcttgccttggatttcagcggcacaaggctcagctgaacctggctgccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgacggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacgctgagcccaaatcggccgac

SEQ ID NO: 7 - protein, 168 aa, HOMO SAPIENS
MACLGFQRHKAQLNLAARTWPCTLLFFLLFIPVFCKAMHVAQPAVVLASSRGIASFVCEYASPGKATEVRVTVLRQADSQVTEVCAATYMTGNELTFLDDSICTGTSSGNQVNLTIQGLRAMDTGLYICKVELMYPPPYYLGIGNGTQIYVIDPEPCPDSDAEPKSAD

SEQ ID NO: 8 - protein, 18 aa, HOMO SAPIENS
PTHVNVSVVMAEVDGTCY

SEQ ID NO: 9 - protein, 6 aa, HOMO SAPIENS
SLSPGK

SEQ ID NO: 10 - protein, 18 aa, HOMO SAPIENS
PTLYNVSLVMSDTAGTCY

SEQ ID NO: 11 - protein, 18 aa, HOMO SAPIENS
PTHVNVSVVMAEVDGTCY

SEQ ID NO: 12 - protein, 10 aa, HOMO SAPIENS
GPSKPEPKSA

SEQ ID NO: 13 - protein, 4 aa, HOMO SAPIENS
NKIL

SEQ ID NO: 14 - protein, 10 aa, HOMO SAPIENS
HFPDQEPKSA

SEQ ID NO: 15 - protein, 4 aa, HOMO SAPIENS
VIHV

SEQ ID NO: 16 - protein, 10 aa, HOMO SAPIENS
PPPDHEPKSA

SEQ ID NO: 17 - protein, 4 aa, HOMO SAPIENS
LKIQ

SEQ ID NO: 18 - protein, 13 aa, HOMO SAPIENS
EPCPDSDAEPKSA

SEQ ID NO: 19 - protein, 4 aa, HOMO SAPIENS
MHVA

SEQ ID NO: 20 - DNA, 21 bp, HOMO SAPIENS
cccaaatcggccgacaaaact

SEQ ID NO: 21 - DNA, 57 bp, HOMO SAPIENS
tcagcgagctctagactacactcatttacccggagacaagcttaggctcttctgcgt

SEQ ID NO: 22 - DNA, 79 bp, HOMO SAPIENS
agcttgtctgcgggtaaacccacccatgtcaatgtgtctgttgtcatggcggaggtggacggcacctgctactgatagt

SEQ ID NO: 23 - DNA, 79 bp, HOMO SAPIENS
ctagactatcagtagcaggtgccgtccacctccgccatgacaacagacacattgacatgggtgggtttacccgcagaca

SEQ ID NO: 24 - protein, 14 aa, HOMO SAPIENS
GPSKPEPKSAGIKP

SEQ ID NO: 25 - protein, 6 aa, HOMO SAPIENS
SLSTGK

SEQ ID NO: 26 - protein, 6 aa, HOMO SAPIENS
SLSAGK

SEQ ID NO: 27 - protein, 5 aa, HOMO SAPIENS
EPKSA